SEQUENCE LISTING (75 sequences)

SEQ ID NO: 1 - DNA, 357 bp, Artificial Sequence
  source: /note="Description of Artificial Sequence: Synthetic polynucleotide"
caggtccaactgcagcagcctggggctgagctggtgaggcctgggtcttcagtgaagctgtcctgcaaggcttctggctacaccttcaccagctactggatggattgggtgaagcagaggcctggacaaggccttgaatggattggtaacatttacccttctgatagtaaaactcactacaatcaaaagttcaaggacaaggccacattgactgtagacaaatcctccagcacagcctacatgcagctcagcagcctgacatctgaggactctgcggtctattactgtgcaagagattactacggtagcatagggatggactactggggtcaaggaacctcagtcaccgtctcctca

SEQ ID NO: 2 - protein, 119 aa, Artificial Sequence
  source: /note="Description of Artificial Sequence: Synthetic polypeptide"
QVQLQQPGAELVRPGSSVKLSCKASGYTFTSYWMDWVKQRPGQGLEWIGNIYPSDSKTHYNQKFKDKATLTVDKSSSTAYMQLSSLTSEDSAVYYCARDYYGSIGMDYWGQGTSVTVSS

SEQ ID NO: 3 - protein, 5 aa, Artificial Sequence
  source: /note="Description of Artificial Sequence: Synthetic peptide"
SYWMD

SEQ ID NO: 4 - protein, 17 aa, Artificial Sequence
  source: /note="Description of Artificial Sequence: Synthetic peptide"
NIYPSDSKTHYNQKFKD

SEQ ID NO: 5 - protein, 10 aa, Artificial Sequence
  source: /note="Description of Artificial Sequence: Synthetic peptide"
DYYGSIGMDY

SEQ ID NO: 6 - DNA, 321 bp, Artificial Sequence
  source: /note="Description of Artificial Sequence: Synthetic polynucleotide"
gacatccagatgacccagtctccatcctccttatctgcctctctgggagaaagagtcagtctcacttgtcgggcaagtcaggaaattagtggttacttaagctggcttcagcagaaaccagatggaactattaaacgcctgatctacgccgcatccactttagattctggtgtcccaaaaaggttcagtggcagtaggtctgggtcagattattctctcaccattagcagccttgagtctgaagattttgcagactattactgtctacaatatgctagttatccgtggacgttcggtggaggcaccaagctggaaatcaaa

SEQ ID NO: 7 - protein, 107 aa, Artificial Sequence
  source: /note="Description of Artificial Sequence: Synthetic polypeptide"
DIQMTQSPSSLSASLGERVSLTCRASQEISGYLSWLQQKPDGTIKRLIYAASTLDSGVPKRFSGSRSGSDYSLTISSLESEDFADYYCLQYASYPWTFGGGTKLEIK

SEQ ID NO: 8 - protein, 11 aa, Artificial Sequence
  source: /note="Description of Artificial Sequence: Synthetic peptide"
RASQEISGYLS

SEQ ID NO: 9 - protein, 7 aa, Artificial Sequence
  source: /note="Description of Artificial Sequence: Synthetic peptide"
AASTLDS

SEQ ID NO: 10 - protein, 9 aa, Artificial Sequence
  source: /note="Description of Artificial Sequence: Synthetic peptide"
LQYASYPWT

SEQ ID NO: 11 - DNA, 357 bp, Artificial Sequence
  source: /note="Description of Artificial Sequence: Synthetic polynucleotide"
caggtccaactgcagcagcctggggctgagctggtgaggcctgggtcttcagtgaagctgtcctgcaaggcttctggctacaccttcaccagctactggatggattgggtgaagcagaggcctggacaaggccttgaatggattggtaacatttacccttctgatagtgcaactcactacaatcaaaagttcaaggacaaggccacattgactatagacaaatcctccagcacagcctacatgcagctcagcagcctgacatctgaggactctgcggtctattactgtgcaagagattactacggtagcatagggatggactcttggggtcaaggaacctctgtcaccgtctcctca

SEQ ID NO: 12 - protein, 119 aa, Artificial Sequence
  source: /note="Description of Artificial Sequence: Synthetic polypeptide"
QVQLQQPGAELVRPGSSVKLSCKASGYTFTSYWMDWVKQRPGQGLEWIGNIYPSDSATHYNQKFKDKATLTIDKSSSTAYMQLSSLTSEDSAVYYCARDYYGSIGMDSWGQGTSVTVSS

SEQ ID NO: 13 - protein, 5 aa, Artificial Sequence
  source: /note="Description of Artificial Sequence: Synthetic peptide"
SYWMD

SEQ ID NO: 14 - protein, 17 aa, Artificial Sequence
  source: /note="Description of Artificial Sequence: Synthetic peptide"
NIYPSDSATHYNQKFKD

SEQ ID NO: 15 - protein, 10 aa, Artificial Sequence
  source: /note="Description of Artificial Sequence: Synthetic peptide"
DYYGSIGMDS

SEQ ID NO: 16 - DNA, 321 bp, Artificial Sequence
  source: /note="Description of Artificial Sequence: Synthetic polynucleotide"
gacatccagatgacccagtctccatcctccttatctgcctctctgggagaaagagtcagtctcacttgtcgggcaagtcaggaaattagtgattacttaagctggcttcagcagaaaccagatggaactattaaacgcctgatctacgccgcatccactttagattctggtgtcccaaaaaggttcagtggcagtaggtctgggtcagattattctctcaccatcagcagccttgagtctgaagattttgcagactattactgtctacaatatgctagttttccgtggacgttcggtggaggcaccaagctggagatcaaa

SEQ ID NO: 17 - protein, 107 aa, Artificial Sequence
  source: /note="Description of Artificial Sequence: Synthetic polypeptide"
DIQMTQSPSSLSASLGERVSLTCRASQEISDYLSWLQQKPDGTIKRLIYAASTLDSGVPKRFSGSRSGSDYSLTISSLESEDFADYYCLQYASFPWTFGGGTKLEIK

SEQ ID NO: 18 - protein, 11 aa, Artificial Sequence
  source: /note="Description of Artificial Sequence: Synthetic peptide"
RASQEISDYLS

SEQ ID NO: 19 - protein, 7 aa, Artificial Sequence
  source: /note="Description of Artificial Sequence: Synthetic peptide"
AASTLDS

SEQ ID NO: 20 - protein, 9 aa, Artificial Sequence
  source: /note="Description of Artificial Sequence: Synthetic peptide"
LQYASFPWT

SEQ ID NO: 21 - DNA, 354 bp, Artificial Sequence
  source: /note="Description of Artificial Sequence: Synthetic polynucleotide"
caggtccaactgcagcagcctggggctgacctggtaaagcctggggcttcagtgaagttgtcctgcaaggcttatggctacactttctttagttactggatgcactgggtgaggcagaggcctggacaaggccttgagtggattggaatgattcatcctaatagtggtaccactaacaacaatgagaagttcaagagcaaggccacactgactgtagacaaatccttcagtacagcctacatgcaactcagcagcctgacatctgaggactctgcggtctattactgtgcgaggggttatagtaactacgactttgactactggggccaaggcaccattctcacagtctcctca

SEQ ID NO: 22 - protein, 118 aa, Artificial Sequence
  source: /note="Description of Artificial Sequence: Synthetic polypeptide"
QVQLQQPGADLVKPGASVKLSCKAYGYTFFSYWMHWVRQRPGQGLEWIGMIHPNSGTTNNNEKFKSKATLTVDKSFSTAYMQLSSLTSEDSAVYYCARGYSNYDFDYWGQGTILTVSS

SEQ ID NO: 23 - protein, 5 aa, Artificial Sequence
  source: /note="Description of Artificial Sequence: Synthetic peptide"
SYWMH

SEQ ID NO: 24 - protein, 17 aa, Artificial Sequence
  source: /note="Description of Artificial Sequence: Synthetic peptide"
MIHPNSGTTNNNEKFKS

SEQ ID NO: 25 - protein, 9 aa, Artificial Sequence
  source: /note="Description of Artificial Sequence: Synthetic peptide"
GYSNYDFDY

SEQ ID NO: 26 - DNA, 321 bp, Artificial Sequence
  source: /note="Description of Artificial Sequence: Synthetic polynucleotide"
gacatcaagatgacccagtttccatcttccatgtatgcttctctaggagagagagtcactatcacttgcaaggcgagtcaggacattaatacctatttaatctggatccagcagaaaccagggaaatctcctaagaccctgatctatcgtgcaaatagattggcagatggggtcccatcaaggttcagtggcagtggatctgggcaagattcttctctcaccatcagcagcctggagcatgaagatatgggaatttattattgtctacagtatgatgagtttccgtatacgttcggaggggggaccaagctggaaataaaa

SEQ ID NO: 27 - protein, 107 aa, Artificial Sequence
  source: /note="Description of Artificial Sequence: Synthetic polypeptide"
DIKMTQFPSSMYASLGERVTITCKASQDINTYLIWIQQKPGKSPKTLIYRANRLADGVPSRFSGSGSGQDSSLTISSLEHEDMGIYYCLQYDEFPYTFGGGTKLEIK

SEQ ID NO: 28 - protein, 11 aa, Artificial Sequence
  source: /note="Description of Artificial Sequence: Synthetic peptide"
KASQDINTYLI

SEQ ID NO: 29 - protein, 7 aa, Artificial Sequence
  source: /note="Description of Artificial Sequence: Synthetic peptide"
RANRLAD

SEQ ID NO: 30 - protein, 9 aa, Artificial Sequence
  source: /note="Description of Artificial Sequence: Synthetic peptide"
LQYDEFPYT

SEQ ID NO: 31 - DNA, 351 bp, Artificial Sequence
  source: /note="Description of Artificial Sequence: Synthetic polynucleotide"
caggttaaacttcaccagtctggggctgaactggcaaaacctggggcctcagtgaagatgtcctgcaaggcctctggctacacctttaatagctactggatacactgggtaaaacagaggcctggacagggtctggaatggatcggatacaataatcctacctctggttatagtgagtacaatcagaagttcacggacaaggccacattgagtgcagacaaatcttccagtacagcctacatgcaactgagcagcctgacatctgaggactctgcagtctattactgtgtaagagagtatggtgacttatttgcttactggggccaagggactctggtcactgtctctgca

SEQ ID NO: 32 - protein, 117 aa, Artificial Sequence
  source: /note="Description of Artificial Sequence: Synthetic polypeptide"
QVKLHQSGAELAKPGASVKMSCKASGYTFNSYWIHWVKQRPGQGLEWIGYNNPTSGYSEYNQKFTDKATLSADKSSSTAYMQLSSLTSEDSAVYYCVREYGDLFAYWGQGTLVTVSA

SEQ ID NO: 33 - protein, 5 aa, Artificial Sequence
  source: /note="Description of Artificial Sequence: Synthetic peptide"
SYWIH

SEQ ID NO: 34 - protein, 17 aa, Artificial Sequence
  source: /note="Description of Artificial Sequence: Synthetic peptide"
YNNPTSGYSEYNQKFTD

SEQ ID NO: 35 - protein, 8 aa, Artificial Sequence
  source: /note="Description of Artificial Sequence: Synthetic peptide"
EYGDLFAY

SEQ ID NO: 36 - DNA, 321 bp, Artificial Sequence
  source: /note="Description of Artificial Sequence: Synthetic polynucleotide"
aacattgtaatgacccaatctcccaaatccatgtccatgtcagtaggagagagggtcaccttgagctgcaaggccagtgagaatgtgggttattctgtatcctggtttcagcagaaaccagatcagtctccaaaactgctgatatacggggcatccaaccggtacactggggtccccgatcgcttcacaggcagtggatctgcaacagatttcactctgaccatcagcactgtgcaggctgaagaccttgcagattatcactgtggacagatttacatctatccgtacacgttcggaggggggaccaagctggaaataaaa

SEQ ID NO: 37 - protein, 107 aa, Artificial Sequence
  source: /note="Description of Artificial Sequence: Synthetic polypeptide"
NIVMTQSPKSMSMSVGERVTLSCKASENVGYSVSWFQQKPDQSPKLLIYGASNRYTGVPDRFTGSGSATDFTLTISTVQAEDLADYHCGQIYIYPYTFGGGTKLEIK

SEQ ID NO: 38 - protein, 11 aa, Artificial Sequence
  source: /note="Description of Artificial Sequence: Synthetic peptide"
KASENVGYSVS

SEQ ID NO: 39 - protein, 7 aa, Artificial Sequence
  source: /note="Description of Artificial Sequence: Synthetic peptide"
GASNRYT

SEQ ID NO: 40 - protein, 9 aa, Artificial Sequence
  source: /note="Description of Artificial Sequence: Synthetic peptide"
GQIYIYPYT

SEQ ID NO: 41 - DNA, 360 bp, Artificial Sequence
  source: /note="Description of Artificial Sequence: Synthetic polynucleotide"
caggcttatctacagcagtctggggctgagctggtgaggcctggggcctcagtgaagatgtcctgcaaggcttctggctacacatttaccagttacaatatgcactgggtaaagcagtcacctagacagggcctggaatgggttggagttttttatttaggaaatggtgatacttcctacaatgagaagttcaagggcaaggccacactgactgtggacaaatcctccagcacagcctacatgcagctcagcagcctgacatctgaagactctgcggtctatttctgtgcaagacccgggggctatgccaactggtacttcgatgtctggggcacagggaccacggtcaccgtctcctca

SEQ ID NO: 42 - protein, 120 aa, Artificial Sequence
  source: /note="Description of Artificial Sequence: Synthetic polypeptide"
QAYLQQSGAELVRPGASVKMSCKASGYTFTSYNMHWVKQSPRQGLEWVGVFYLGNGDTSYNEKFKGKATLTVDKSSSTAYMQLSSLTSEDSAVYFCARPGGYANWYFDVWGTGTTVTVSS

SEQ ID NO: 43 - protein, 5 aa, Artificial Sequence
  source: /note="Description of Artificial Sequence: Synthetic peptide"
SYNMH

SEQ ID NO: 44 - protein, 17 aa, Artificial Sequence
  source: /note="Description of Artificial Sequence: Synthetic peptide"
VFYLGNGDTSYNEKFKG

SEQ ID NO: 45 - protein, 11 aa, Artificial Sequence
  source: /note="Description of Artificial Sequence: Synthetic peptide"
PGGYANWYFDV

SEQ ID NO: 46 - DNA, 321 bp, Artificial Sequence
  source: /note="Description of Artificial Sequence: Synthetic polynucleotide"
gacattgtgatgacccagtctcaaaaattcatgtccacatcagtaggagactgggtcagcgtcacctgcaaggccagtcagaatgtgggtactgaggtatcctggtatcaacagaaaccaggccaatctcctaatacactgatctactcggcatcctatcggcacagtggagtccctgatcgcttcacaggcagtggatctgggacagaattcactctcaccatcaccaatgtgcagtctgaagacttggcagactatttctgtcagcaatataacagctttccgctcacgttcggtgctgggaccaagctggacctgaaa

SEQ ID NO: 47 - protein, 107 aa, Artificial Sequence
  source: /note="Description of Artificial Sequence: Synthetic polypeptide"
DIVMTQSQKFMSTSVGDWVSVTCKASQNVGTEVSWYQQKPGQSPNTLIYSASYRHSGVPDRFTGSGSGTEFTLTITNVQSEDLADYFCQQYNSFPLTFGAGTKLDLK

SEQ ID NO: 48 - protein, 11 aa, Artificial Sequence
  source: /note="Description of Artificial Sequence: Synthetic peptide"
KASQNVGTEVS

SEQ ID NO: 49 - protein, 7 aa, Artificial Sequence
  source: /note="Description of Artificial Sequence: Synthetic peptide"
SASYRHS

SEQ ID NO: 50 - protein, 9 aa, Artificial Sequence
  source: /note="Description of Artificial Sequence: Synthetic peptide"
QQYNSFPLT

SEQ ID NO: 51 - protein, 117 aa, Artificial Sequence
  source: /note="Description of Artificial Sequence: Synthetic polypeptide"
EVQLVQSGAEVKKPGSSVKVSCKASGYTFNSYWIHWVRQAPGQGLEWMGYNNPTSGYSEYNQKFTDRVTITADKSTSTAYMELSSLRSEDTAVYYCAREYGDLFAYWGQGTLVTVSS

SEQ ID NO: 52 - protein, 107 aa, Artificial Sequence
  source: /note="Description of Artificial Sequence: Synthetic polypeptide"
AIQMTQSPSSLSASVGDRVTITCKASENVGYSVSWFQQKPGKSPKLLIYGASNRYTGVPSRFSGSGSATDFTLTISSLQPEDFATYHCGQIYIYPYTFGGGTKVEIK

SEQ ID NO: 53 - DNA, 351 bp, Artificial Sequence
  source: /note="Description of Artificial Sequence: Synthetic polynucleotide"
gaggtgcagctggtgcagagcggcgccgaagtgaaaaagcccggcagcagcgtgaaggtcagctgcaaggcctccgggtacaccttcaacagctactggatccactgggtgaggcaggcccccggccagggcctcgagtggatgggctacaacaaccccaccagcggctacagcgagtacaaccagaagttcaccgacagggtgaccatcacagccgacaagagcaccagcaccgcctacatggagctgagcagcctgaggagcgaggacaccgccgtgtattactgcgcaagggagtacggcgacctgttcgcctactggggccagggcaccctggtgaccgtgagctct

SEQ ID NO: 54 - DNA, 321 bp, Artificial Sequence
  source: /note="Description of Artificial Sequence: Synthetic polynucleotide"
gccatccagatgacccagagccctagcagcctgagcgccagcgtgggagacagggtgaccatcacctgcaaggccagcgagaacgtgggctacagcgtgagctggttccagcagaagcccggcaagagccccaagctgctgatctacggcgcaagcaacaggtacaccggcgtgccctctaggtttagcggcagcggcagcgccaccgacttcaccctgaccatcagcagcctccagcccgaggacttcgccacctaccactgcggccagatctacatctacccctacactttcggcggcggcaccaaggtggagattaag

SEQ ID NO: 55 - protein, 447 aa, Artificial Sequence
  source: /note="Description of Artificial Sequence: Synthetic polypeptide"
EVQLVQSGAEVKKPGSSVKVSCKASGYTFNSYWIHWVRQAPGQGLEWMGYNNPTSGYSEYNQKFTDRVTITADKSTSTAYMELSSLRSEDTAVYYCAREYGDLFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 214 aa, Artificial Sequence
  source: /note="Description of Artificial Sequence: Synthetic polypeptide"
AIQMTQSPSSLSASVGDRVTITCKASENVGYSVSWFQQKPGKSPKLLIYGASNRYTGVPSRFSGSGSATDFTLTISSLQPEDFATYHCGQIYIYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - DNA, 1344 bp, Artificial Sequence
  source: /note="Description of Artificial Sequence: Synthetic polynucleotide"
gaagtgcagctggtgcagagcggagcagaagtgaagaagcccggaagcagcgtgaaggtgtcttgcaaggccagcggctacaccttcaacagctactggatccattgggtgcgccaggctccaggacagggactcgagtggatgggatacaacaaccccaccagcggctacagcgagtacaaccagaagttcaccgaccgcgtgaccatcacagccgataagagcaccagcaccgcctacatggagctgtctagcctgaggagcgaggacacagccgtgtactattgcgcccgggagtacggagacctgttcgcttattggggccagggaacactggtgacagtgtccagcgcttcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 58 - DNA, 645 bp, Artificial Sequence
  source: /note="Description of Artificial Sequence: Synthetic polynucleotide"
gccatccagatgacccagtctcctagcagcctgagcgccagcgtgggagatagagtgaccatcacttgcaaggccagcgagaacgtgggctacagcgtgtcttggttccagcagaagcccggcaagagccctaagctgctgatctacggcgcctctaacagatacaccggcgtgcctagcagattcagcggcagcggaagcgccacagacttcaccctgaccatcagcagcctgcagccagaagacttcgccacctaccattgcggccagatctacatctacccctacaccttcggcggaggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 59 - protein, 570 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 60 - protein, 120 aa, Artificial Sequence
  source: /note="Description of Artificial Sequence: Synthetic polypeptide"
QVQLVQSGAEVKKPGASVKVSCKASGYTFGSGGISWVRQAPGQGLEWMGWISDYNGQTNYAQKLQGRVTMTTDTSTSTAYMELRSLRSDDTAVYYCARVGTDTWYAFDIWGQGTLVTVSS

SEQ ID NO: 61 - protein, 106 aa, Artificial Sequence
  source: /note="Description of Artificial Sequence: Synthetic polypeptide"
EIVLTQSPATLSLSPGERATLSCRASQSVSSYLAWYQQKPGQAPRLLIYDASNRATGIPARFSGSGSGTDFTLTISSLEPEDFAVYYCQQPDNLRTFGGGTKVEIK

SEQ ID NO: 62 - DNA, 357 bp, Artificial Sequence
  source: /note="Description of Artificial Sequence: Synthetic polynucleotide"
gaggtccagctgcaacaatctggacctgagctggtgaagcctggggcttcagtgaagatatcctgtaaggcttctggatacacgttcactgactactacatgaactgggtgaaacagagccatggaaagagccttgagtggattggacatattaatcctaacagcggtagtactacctacactgagaagttcaaggacaaggccacattgactgtagacaagtcctccagcacagcctacatggagctccgcagcctgacatctgacgactctgcagtctattactgtgtcagaaggattgggaagaactggcattgcgatgtctggggcacagggaccacggtcaccgtctcctca

SEQ ID NO: 63 - protein, 119 aa, Artificial Sequence
  source: /note="Description of Artificial Sequence: Synthetic polypeptide"
EVQLQQSGPELVKPGASVKISCKASGYTFTDYYMNWVKQSHGKSLEWIGHINPNSGSTTYTEKFKDKATLTVDKSSSTAYMELRSLTSDDSAVYYCVRRIGKNWHCDVWGTGTTVTVSS

SEQ ID NO: 64 - protein, 5 aa, Artificial Sequence
  source: /note="Description of Artificial Sequence: Synthetic peptide"
DYYMN

SEQ ID NO: 65 - protein, 17 aa, Artificial Sequence
  source: /note="Description of Artificial Sequence: Synthetic peptide"
HINPNSGSTTYTEKFKD

SEQ ID NO: 66 - protein, 10 aa, Artificial Sequence
  source: /note="Description of Artificial Sequence: Synthetic peptide"
RIGKNWHCDV

SEQ ID NO: 67 - DNA, 333 bp, Artificial Sequence
  source: /note="Description of Artificial Sequence: Synthetic polynucleotide"
gacattgtgctgacccaatctccagcttctttggctgtgtctctagggcagagggccaccatctcctgcagagccagtgaaagtgtcaactttcatggtactcatttaatgcactggtaccaacagaaaccaggacaggcacccaaactcctcatctatgctgcatccaacctagattctggagtccctgccaggttcagtggcagtgggtctgagacagacttcaccctcaacatccatcctgtggaggaggaggatgctgcgacctatttctgtcagcaaagtattgaggatccattcacgttcggctcggggacatacttggaaataaaa

SEQ ID NO: 68 - protein, 111 aa, Artificial Sequence
  source: /note="Description of Artificial Sequence: Synthetic polypeptide"
DIVLTQSPASLAVSLGQRATISCRASESVNFHGTHLMHWYQQKPGQAPKLLIYAASNLDSGVPARFSGSGSETDFTLNIHPVEEEDAATYFCQQSIEDPFTFGSGTYLEIK

SEQ ID NO: 69 - protein, 15 aa, Artificial Sequence
  source: /note="Description of Artificial Sequence: Synthetic peptide"
RASESVNFHGTHLMH

SEQ ID NO: 70 - protein, 7 aa, Artificial Sequence
  source: /note="Description of Artificial Sequence: Synthetic peptide"
AASNLDS

SEQ ID NO: 71 - protein, 9 aa, Artificial Sequence
  source: /note="Description of Artificial Sequence: Synthetic peptide"
QQSIEDPFT

SEQ ID NO: 72 - DNA, 351 bp, Artificial Sequence
  source: /note="Description of Artificial Sequence: Synthetic polynucleotide"
gaagtgcagctggtgcagagcggagcagaagtgaagaagcccggaagcagcgtgaaggtgtcttgcaaggccagcggctacaccttcaacagctactggatccattgggtgcgccaggctccaggacagggactcgagtggatgggatacaacaaccccaccagcggctacagcgagtacaaccagaagttcaccgaccgcgtgaccatcacagccgataagagcaccagcaccgcctacatggagctgtctagcctgaggagcgaggacacagccgtgtactattgcgcccgggagtacggagacctgttcgcttattggggccagggaacactggtgacagtgtccagc

SEQ ID NO: 73 - protein, 119 aa, Artificial Sequence
  source: /note="Description of Artificial Sequence: Synthetic polypeptide"
EVQLVQSGAEVKKPGESLKISCKGSGYSFNSRWIGWVRQMPGKGLEWMGIIYPGDSDVRYSPSFQGQVTISADKSISTAYLQWSSLKASDTAVYYCARVGGGILYMDVWGKGTTVTVSS

SEQ ID NO: 74 - protein, 108 aa, Artificial Sequence
  source: /note="Description of Artificial Sequence: Synthetic polypeptide"
EIVMTQSPATLSVSPGERATLSCRASQSVSSNLAWYQQKPGQAPRLLIYGASTRATGIPARFSGSGSGTEFTLTISSLQSEDFAVYYCQQSYIWPPITFGGGTKVEIK

SEQ ID NO: 75 - protein, 119 aa, Artificial Sequence
  source: /note="Description of Artificial Sequence: Synthetic polypeptide"
EVQLQQSGPELVKPGASVKISCKASGYTFTDYYMNWVKQSHGKSLEWIGHINPNSGSTTYTEKFKDKATLTVDKSSSTAYMELRSLTSDDSAVYYCVRRIGKNWHSDVWGTGTTVTVSS

(19) United States
(12) Patent Application Publication
(10) Pub. No.: US 2023/0295313 A1
(43) Pub. Date: Sep. 21, 2023

(54) IL1RAP BINDING PROTEINS

(71) Applicant: GLAXOSMITHKLINE INTELLECTUAL PROPERTY DEVELOPMENT LIMITED, Brentford Middlesex (GB)

(72) Inventors: John G. Emery, Collegeville, PA (US); Qi Fei, Shanghai (CN); Shiyong Gong, Shanghai (CN); Sanjay Kumar, Collegeville, PA (US); Fang Ren, Shanghai (CN); Teddy Yang, Shanghai (CN); Hua Ying, Shanghai (CN)

(73) Assignee: GLAXOSMITHKLINE INTELLECTUAL PROPERTY DEVELOPMENT LIMITED, Brentford Middlesex (GB)

(21) Appl. No.: 17/622,689

(22) PCT Filed: Jun. 22, 2020
(86) PCT No.: PCT/IB2020/055887

§ 371 Date: Dec. 23, 2021

(30) Foreign Application Priority Data
Jun. 26, 2019 (WO) PCT/CN2019/093114

(51) Int. Cl. C07K 16/28; A61P 35/00; C12N 15/63

(52) U.S. Cl. CPC C07K 16/2866; A61P 35/00; C12N 15/63; C07K 2317/24; C07K 2317/41; C07K 2317/565; C07K 2317/732; C07K 2317/734

(57) ABSTRACT

The present invention relates generally to antibodies in the treatment of human disease and reduction of adverse events related thereto. More specifically, the present invention relates to the use of IL 1RAP (Interleukin-1 receptor accessory protein) binding proteins (including anti-IL 1RAP 0 antagonist antibodies) and their use as treatment and prevention of human disease.

### CROSS REFERENCE TO RELATED APPLICATIONS

This application is a national stage application under 35 U.S.C. § 371 of International Patent Application No. PCT/IB2020/055887, filed Jun. 22, 2020, which claims priority from International Patent Application No. PCT/CN2019/093114, filed Jun. 26, 2019. The disclosure of each of the aforementioned prior applications is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 18, 2020, is named PU66378_SL.txt and is 54,788 bytes in size.

### FIELD OF THE INVENTION

The present invention relates generally to antibodies in the treatment of human disease and reduction of adverse events related thereto. More specifically, the present invention relates to the use of IL1RAP (Interleukin-1 receptor accessory protein) binding proteins (including anti-IL1RAP antagonist antibodies) and their use as treatment and prevention of human disease.

### BACKGROUND OF THE INVENTION

Acute myeloid leukemia (AML), chronic myeloid leukemia (CML), and myelodysplastic syndrome (MDS) are characterized by aberrant clonal proliferation and expansion of immature myeloid precursors, resulting in dysfunctional hematopoietic cell differentiation. AML is the most common leukemia diagnosed in adults with an incidence in the US and Europe ranging from 3 to 5 per 100,000. The incidence of AML increases with age, and overall survival ranges from 6 months to 12 years. Curative treatment consists of high dose cytotoxic chemotherapy and/or bone marrow transplantation, which is primarily restricted to younger patients (< 60 years of age) because of the toxicity of the regimen and the relative chemo-resistance of the disease in older adults. Therefore, AML in older adults represents a significant unmet clinical need for which therapies with an improved risk/benefit profile are desirable (Khwaja 2016, Nature Reviews Disease Primers vol 2). CML is characterized by a chromosomal translocation, the Philadelphia (Ph) chromosome, leading to the fusion gene BCR-ABL. The resultant fusion protein, the BCR-ABL protein kinase, is constitutively activated and leads to uncontrolled proliferation. The incidence of CML is 1.75 per 100,000 individuals, which also increases with age. Although in many CML patients the disease is effectively managed with tyrosine kinase inhibitors, there exists a subset of patients who fail to respond or develop resistance to marketed therapeutics, and thus represent an unmet medical need. (Storey 2009 Nat Rev Drug Disc 8:447; Chen et al., 2013, Leukemia & Lymphoma 54:1411). MDS is a group of chronic myeloid neoplasms that result in peripheral blood cytopenias and functional blood cell deficiencies, which, in some cases, can progress to AML (Steensa 2015, Mayo Clin Proc 90:969).

The goal of chemotherapeutic regimens for AML is to drive the disease into a long-standing durable remission. However, in many cases, the disease will recur within several months to several years. Disease recurrence is thought to be due to the persistence of leukemic stem cells (LSC), slowly dividing cells akin to hematopoietic stem cells, but which give rise to a mutant leukemic cell clone. LSCs have been shown to express elevated levels of several cell surface molecules, such as CD123, CD33, and CLL-1, and it has been proposed that this presents an opportunity to eradicate the disease by targeting and depleting LSCs with monoclonal antibodies (mAb) that bind LSC surface markers (Pollyea 2017, Blood 23:1627). Interleukin-1 Receptor Accessory Protein (IL1RAP), a co-receptor for IL-1, IL-33 and IL-36, was identified as a cell surface marker that is elevated on LSCs (Jaras 2010, PNAS 107:16280; Barreyro 2012, Blood 120:1290; Jiang 2014, Cancer Res 2014;74(19 Suppl):Abstract 662). Additionally, monoclonal antibodies against IL1RAP have been shown to kill AML cells in vitro by antibody-dependent cellular cytotoxicity (ADCC) and inhibit AML and CML cell engraftment in murine xenotransplantation models (Askmyr 2013, Blood 121:3709; Agerstam 2015, PNAS 112: 10786; Agerstam 2016, Blood 128:2683).

IL1RAP also plays an important functional role in hematologic malignancies by facilitating growth-promoting signal transduction by IL-1. In a recent study, IL-1 stimulated the expansion of most primary AML patient samples while suppressing the growth of normal progenitor cells (Carey 2017, Cell Reports 18, 3204-3218). In addition, IL-1 promoted a leukemogenic environment in CML, promoting the expansion of CML stem cells but not that of healthy hematopoietic stem cells (Ågerstam 2016, Blood 128:2683). Furthermore, growth of CML cells was blocked by the IL-1 receptor antagonist protein (IL-1RA), suggesting that an IL-1 neutralizing IL1RAP mAb would have similar activity (Zhang 2016, Blood 128:2671). A role for IL1RAP in leukemia is also supported by a retrospective analysis of clinical data suggesting diminished survival for AML patients with high IL1RAP expression, thereby strongly implicating IL1RAP in AML disease progression (Barreyro 2012, Blood 120:1290). Additionally, inhibition of IL-1 function reduces AML and CML cell proliferation or colony formation in vitro (Cohen 1991, Cozzolina 1989, Sissolak 1992, Rambaldi 1991, Estrov 1992, Estrov 1994, Stosic-Grujicic 1995, Estrov 1991, Bagby 1988, Schiro 1993). Collectively, these data suggest that antibodies directed against IL1RAP could inhibit cell growth in various hematologic malignancies.

In addition to its role in hematologic cancers, IL-1 is implicated in the growth, metastasis, and immune suppression of multiple solid tumors (e.g., NSCLC, breast, melanoma) (reviewed in Apte 2017, Journal of Leukocyte Biology 102:293). Data from The Cancer Genome Atlas (TCGA, http://cancergenome.nih.gov/) and the Cancer Cell Line Encyclopedia (CCLE, www.broadinstitute.org/ccle) indicate that IL1RAP is expressed in multiple solid tumors, and IL1RAP targeted mAbs exhibit potent in vitro ADCC activity on solid tumor cell lines and have demonstrated efficacy in multiple murine xenograft models of solid tumors (e.g., melanoma, NSCLC) (FIG. 4, WO12098407, US2017121420). Blockade of the IL-1 pathway by genetic manipulation or use of IL-1 receptor antagonist (IL-1RA) inhibits the growth and metastasis of solid tumors, overcomes kinase inhibitor resistance, and enhances the effect of chemotherapy (Bruchard 2013, Nat Med 19:57-64, Guo 2016, Scientific Reports 6:srep36107, Holen 2016, Oncotarget 7:75571-75584, Song 2005, The Journal of Immunology 175:8200, Stanam 2016, Oncotarget 7:76087-76100, Watari 2014, PLOS ONE 9:e99568).

Multiple studies indicate that IL-1 promotes a tumor-permissive environment through myeloid-derived suppressor cells (MDSCs) (Guo 2016, Scientific Reports 6:srep36107, Song 2005, The Journal of Immunology 175:8200, Mager 2016, Frontiers in Oncology 6, doi:10.3389), and an IL1RAP-CD3 bispecific mAb capable of depleting MDSC in cancer patient blood has shown efficacy in NSCLC xenograft models (US2017121420). Importantly, a connection between inhibition of IL-1β and lung cancer was recently demonstrated in patients in a large Phase 3 atherosclerosis trial (CANTOS), in which a cancer subset analysis demonstrated that the IL-1β blocking mAb, canakinumab, reduced the incidence and mortality of lung cancer (Ridker 2017, The Lancet, doi:10.1016). In contrast to an IL-1β blocking mAb, an Fc-enhanced IL1RAP mAb that inhibits IL-1 signaling at the receptor level would have the added benefit of inhibiting both IL-1α and IL-1β, as well as killing IL1RAP expressing cancer cells or MDSCs by ADCC.

### SUMMARY OF THE INVENTION

The present invention generally relates to ADCC-enabled IL-1 neutralizing IL1RAP antibodies having therapeutic efficacy in AML as well as multiple solid tumors (cancers such as, but not limited to, prostate, breast, lung, colon, melanoma, bladder, brain, cervical, esophageal, gastric, head/neck, kidney, liver, ovarian, lymphoma, pancreatic, and sarcomas) by directly killing IL1RAP expressing tumor cells via ADCC and/or CDC, inhibiting the growth promoting effect of IL-1 on cancer cells, and promoting an enhanced tumor-directed immune response by inhibiting the MDSC-promoting effects of IL-1, or directly killing IL1RAP expressing MDSC.

In one embodiment, IL1RAP binding proteins (including anti-IL1RAP antibodies) are provided which competes for binding to IL1RAP proteins with any one of the IL1RAP antibodies having heavy and light variable regions comprising respective heavy and light variable sequences of: SEQ ID NO:2 and 7; 12 and 17; 22 and 27; 32 and 37; 42 and 47; 51 and 52; or 63 and 68.

In one embodiment, IL1RAP binding proteins (including anti-IL1RAP antibodies) are provided which competes for binding to IL1RAP proteins with any one of the IL1RAP antibodies having heavy and light variable regions comprising respective heavy and light variable sequences of: SEQ ID NO:2 and 7; 12 and 17; 22 and 27; 32 and 37; 42 and 47; 51 and 52; or 63 and 68, and furthermore said IL1RAP binding proteins have the ability to directly kill IL1RAP expressing tumor cells via ADCC and/or CDC, inhibit the growth promoting effect of IL-1 on cancer cells, and promote an enhanced tumor-directed immune response by inhibiting the MDSC-promoting effects of IL-1, or directly killing IL1RAP expressing MDSC.

In one embodiment, IL1RAP binding proteins (including anti-IL1RAP antibodies) are provided that binds to human IL1RAP at one or both amino acids at Gln165 and Asn166 of human IL1RAP of SEQ ID NO 59 as determined by X-ray crystallography.

In one embodiment, IL1RAP binding proteins (including anti-IL1RAP antibodies) are provided that contact human IL1RAP at one or both amino acids at Gln165 and Asn166 of human IL1RAP of SEQ ID NO 59 within 5 Angstroms as determined by X-ray crystallography.

In one embodiment of the present invention, IL1RAP binding proteins are provided comprising one or more of: CDRH1 as set forth in SEQ ID NO:33; CDRH2 as set forth in SEQ ID NO:34; CDRH3 as set forth in SEQ ID NO:35; CDRL1 as set forth in SEQ ID NO:38; CDRL2 as set forth in SEQ ID NO:39 and/or CDRL3 as set forth in SEQ ID NO:40; or a direct equivalent of each CDR wherein a direct equivalent has up to two amino acid conservative substitutions in said CDR; or having up to 2 amino acid deletions or additions within any CDR.

In one embodiment of the present invention, IL1RAP binding proteins are provided which specifically binds to human IL1RAP of SEQ ID NO: 59 wherein said IL1RAP binding protein comprises a V_{H} domain comprising an amino acid sequence at least 85, 90, 95, 98, or 99% identical to the amino acid sequence set forth in SEQ ID NO:51 and/or a V_{L} domain comprising an amino acid sequence at least 85, 90, 95, 98, or 99% identical to the amino acid sequence set forth in SEQ ID NO:52.

In one embodiment of the present invention, IL1RAP binding proteins are provided which specifically binds to human IL1RAP of SEQ ID NO: 59 wherein said IL1RAP binding protein comprises a V_{H} domain comprising an amino acid sequence at least 85, 90, 95, 98, or 99% identical to the amino acid sequence set forth in SEQ ID NO:73 and/or a V_{L} domain comprising an amino acid sequence at least 85, 90, 95, 98, or 99% identical to the amino acid sequence set forth in SEQ ID NO:74.

In one embodiment, humanized monoclonal antibodies are provided comprising heavy chain CDRs having the amino acid sequences set forth in SEQ ID NO:33; SEQ ID NO:34; and SEQ ID NO:35, and light chain CDRs having the amino acid sequences set forth in SEQ ID NO:38; SEQ ID NO:39; and SEQ ID NO:40. In one embodiment, humanized monoclonal antibodies are provided which comprise a V_{H} domain comprising an amino acid sequence set forth in SEQ ID NO:51; and a V_{L} domain comprising an amino acid sequence set forth in SEQ ID NO:52. In one embodiment, humanized monoclonal antibodies are provided which comprise full length heavy chain comprising an amino acid sequence set forth in SEQ ID NO:55; and full length light chain comprising an amino acid sequence set forth in SEQ ID NO:56.

In one embodiment, the present invention provides antibody GSK3903371A.

In one embodiment, the present invention provides an antibody having VH and VL polypeptide sequences of SEQ ID NO: 73 and 74, respectively.

In one embodiment, the present invention relates to a method of treating acute myeloid leukemia (AML), chronic myeloid leukemia (CML), and myelodysplastic syndrome (MDS); and solid tumor cancers selected from the group consisting of prostate, breast, lung, colon, melanoma, bladder, brain, cervical, esophageal, gastric, head/neck, kidney, liver, ovarian, lymphoma, pancreatic, and sarcomas in a human comprising administering therapeutically effective amount of any one of IL1RAP binding proteins (including anti-IL1RAP antibodies) described above.

In one embodiment, the present invention relates to a method of treating diseases associated with inflammatory cytokines (such as IL1alpha, IL1beta, IL33, IL36), including rheumatoid arthritis, gout, psoriasis, hidradenitis suppurativa, allergic inflammation, asthma, atopic dermatitis, in a human comprising administering therapeutically effective amount of any one of IL1RAP binding proteins (including anti-IL1RAP antibodies) described above.

In one embodiment of the present invention, polynucleotides encoding IL1RAP binding proteins are provided said binding proteins comprise one or more of: CDRH1 as set forth in SEQ ID NO:33; CDRH2 as set forth in SEQ ID NO:34; CDRH3 as set forth in SEQ ID NO:35; CDRL1 as set forth in SEQ ID NO:38; CDRL2 as set forth in SEQ ID NO:39 and/or CDRL3 as set forth in SEQ ID NO:40 or a direct equivalent of each CDR wherein a direct equivalent has no more than two amino acid substitutions in said CDR.

In one embodiment of the present invention, polynucleotides encoding IL1RAP binding proteins are provided said binding proteins specifically bind to human IL1RAP of SEQ ID NO:59, and wherein said IL1RAP binding protein comprises a V_{H} domain comprising an amino acid sequence at least 85, 90, 95, 98, or 99% identical to the amino acid sequence set forth in SEQ ID NO:51 and/or a V_{L} domain comprising an amino acid sequence at least 85, 90, 95, 98, or 99% identical to the amino acid sequence set forth in SEQ ID NO:52.

In one embodiment, polynucleotides encoding humanized monoclonal antibodies are provided said antibodies comprise heavy chain CDRs having the amino acid sequences set forth in SEQ ID NO:33; SEQ ID NO:34; and SEQ ID NO:35 and light chain CDRs having the amino acid sequences set forth in SEQ ID NO:38; SEQ ID NO:39; and SEQ ID NO:40. In one embodiment, polynucleotides encoding humanized monoclonal antibodies are provided said antibodies comprise a V_{H} domain comprising an amino acid sequence set forth in SEQ ID NO:51; and a V_{L} domain comprising an amino acid sequence set forth in SEQ ID NO:52. In one embodiment, polynucleotides encoding humanized monoclonal antibodies are provided said antibodies comprise full length heavy chain comprising an amino acid sequence as set forth in SEQ ID NO:55; and full length light chain comprising an amino acid sequence set forth in SEQ ID NO:56.

In one embodiment, polynucleotides comprising polynucleotides having identity at least 85, 90, 95, 98, or 99% to polynucleotide of SEQ ID NO:53, 54, 57, 58 or 72 are provided.

In one embodiment, a polynucleotide comprising SEQ ID NO:53, 54, 57, 58, or 72 is provided.

In one embodiment, a host cell and a vector containing a polynucleotide comprising a polynucleotide having identity at least 85, 90, 95, 98, or 99% to polynucleotide of SEQ ID NO:53, 54, 57, 58 or 72 are provided.

In one embodiment, methods are provided for treating acute myeloid leukemia (AML), chronic myeloid leukemia (CML), and myelodysplastic syndrome (MDS); and solid tumor cancers selected from the group consisting of prostate, breast, lung, colon, melanoma, bladder, brain, cervical, esophageal, gastric, head/neck, kidney, liver, ovarian, lymphoma, pancreatic, and sarcomas, and diseases associated with inflammatory cytokines (such as IL1alpha, IL1beta, IL33, or IL36), such as, and not limited to, rheumatoid arthritis, gout, psoriasis, hidradenitis suppurativa, allergic inflammation, asthma, atopic dermatitis, with a pharmaceutical composition comprising at least one IL1RAP binding protein of the present invention as described above.

### BRIEF DESCRIPTION OF THE DRAWINGS

FIG. 1. X-ray figure for mAb063 murine Fab binding to IL1RAP protein. Numbers indicate amino acid residues on IL1RAP which approach to mAb063 within 3.5 Angstrom.

FIG. 2. X-ray figure for mAb067 murine Fab binding to IL1 protein. Numbers indicate amino acid residues on IL1RAP which approach to mAb067 within 3.5 Angstrom.

FIG. 3. X-ray figure for mAb154F01-16 Fab binding to IL1 protein. Numbers indicate amino acid residues on IL1RAP which approach mAb067 within 3.5 Angstrom.

FIG. 4. Diagram of how binning studies are done.

FIG. 5. Format of surface plasmon resonance (SPR) assay for determining antibody binding kinetics and affinity.

FIG. 6. Cell killing activity of GSK3903371A in a PBMC ADCC assay with OCI-AML-1 target cells (n=3, representative plot from experiment 1).

FIG. 7. The ADCC activity of afucosylated anti-IL1RAP mAb GSK3903371A is more potent than the humanized mAb067-12 with primary AML target cells.

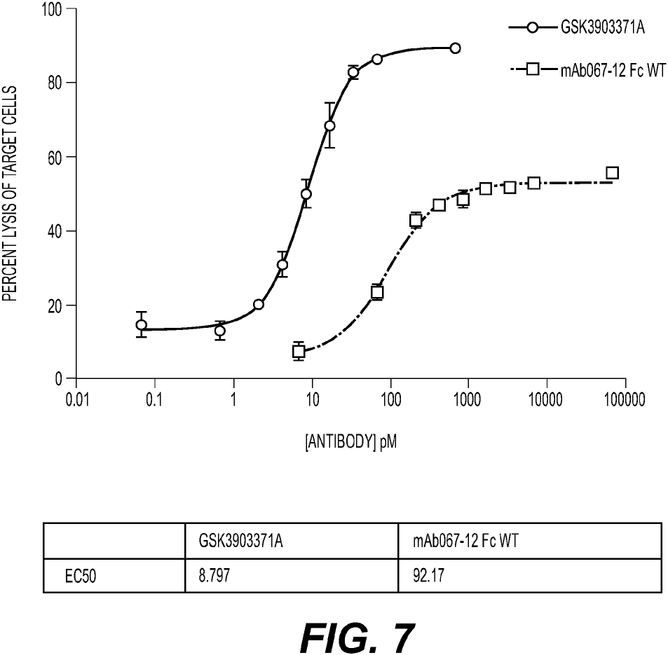

FIG. 8. CDC activity of humanized mAb067-12 and GSK3903371A. Humanized mAb067-12 and GSK3903371A were evaluated in CDC assays using HEKK293/IL1RAP cells. Anti-RAC.huIgG1 is a negative control IgG1 mAb that recognizes an irrelevant antigen.

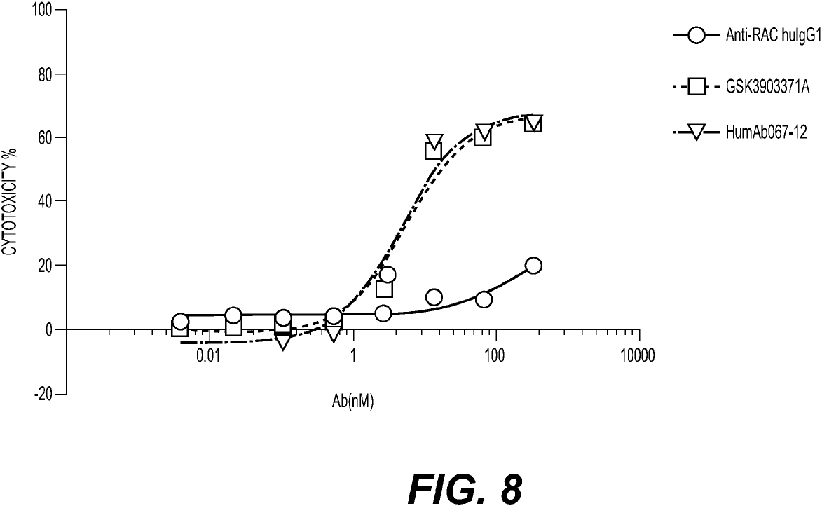

FIG. 9. GSK3903371A inhibits growth of a human AML PDX line in immunodeficient NOG mice.

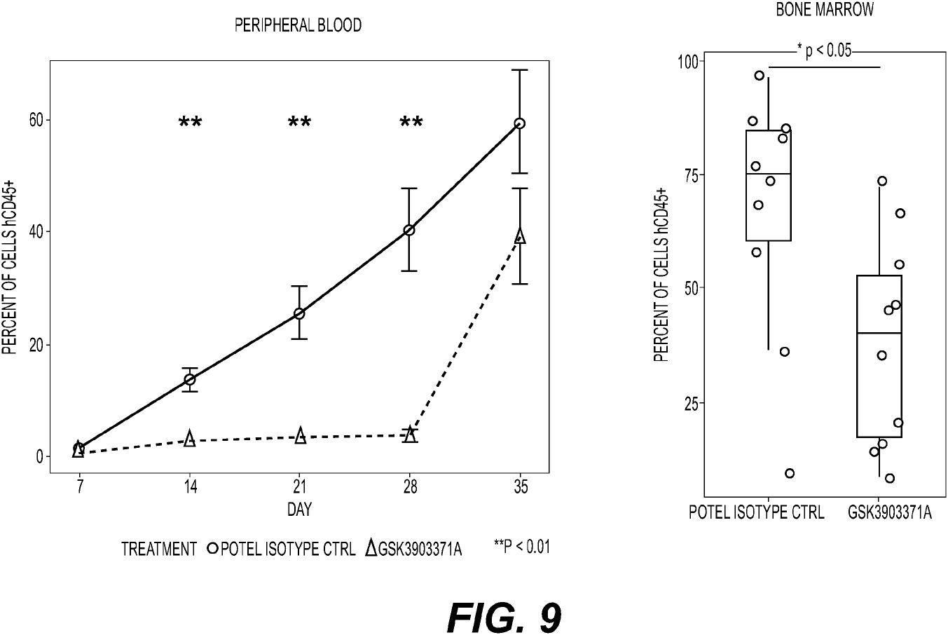

### DETAILED DESCRIPTION OF THE INVENTION

### Definitions

As used herein “IL1RAP protein” or “IL1RAP” is Interleukin-1 receptor accessory protein. The amino acid sequence of human IL1RAP is provided as SEQ ID NO:59.

As used herein the term “competes for binding” refers to any IL1RAP binding protein (including antibodies) that will compete for binding to IL1RAP with any of the IL1RAP binding proteins (including antibodies) of the present invention. Competition for binding between two molecules for IL1RAP can be tested by various methods known in the art including Flow cytometry, Meso Scale Discovery and ELISA. Binding can be measured directly, meaning two or more binding proteins can be put in contact with IL1RAP and binding may be measured for one or each. Alternatively, binding of molecules of interest can be tested against the binding of natural ligand and quantitatively compared with each other. Competition for binding can also be measured using the technique of binning which is standard technique in the art, such as binning studies as described below.

The IL1RAP binding protein of the present invention binds to IL1RAP protein. In one embodiment, the term “binds” or “binding to” means that at least one or more amino acid residues of IL1RAP binding protein approaches within ≤5.0, ≤4.5, ≤4.0 or ≤3.5 Angstrom to at least one or more amino acid residues of IL1RAP protein, often as measured by X-ray crystallography. When the IL1RAP binding protein is an antibody, more often some of the amino acid residues within CDRs or framework regions of the antibody approaches within ≤5.0, ≤4.5, ≤4.0 or ≤3.5 Angstrom of at least one or more amino acid residues of IL1RAP protein. Also, the term “binds” or “binding to” also can mean that the interaction of IL1RAP binding protein (including an anti-IL1RAP antibody) and IL1RAP ligand has a KD (equilibrium dissociation constant) value of less than 1×10⁻⁸, 1×10⁻⁹, even more preferably 1×10⁻¹⁰ nM.

The term “IL1RAP binding protein” as used herein refers to antibodies, fragments thereof, and other protein constructs, such as domains, which are capable of binding to IL1RAP. In some instances, the IL1RAP is human IL1RAP. The term “IL1RAP binding protein” can be used interchangeably with “IL1RAP antigen binding protein.” Thus, as is understood in the art, anti-IL1RAP antibodies and/or IL1RAP antigen binding proteins would be considered IL1RAP binding proteins. As used herein, “antigen binding protein” is any protein, including but not limited to antibodies, domains and other constructs described herein, that binds to an antigen, such as IL1RAP. As used herein “antigen binding portion” of an IL1RAP binding protein would include any portion of the IL1RAP binding protein capable of binding to IL1RAP, including but not limited to, an antigen binding antibody fragment.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., V_{H}, V_{HH}, V_{L}, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer or an EGF domain.

The term “domain” refers to a folded protein structure which retains its tertiary structure independent of the rest of the protein. Generally, domains are responsible for discrete functional properties of proteins and in many cases may be added, removed or transferred to other proteins without loss of function of the remainder of the protein and/or of the domain.

The term “single variable domain” refers to a folded polypeptide domain comprising sequences characteristic of antibody variable domains. It therefore includes complete antibody variable domains such as V_{H}, V_{HH} and V_{L}, and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “dAb(™)” may be considered the same as a “single variable domain”. A single variable domain may be a human single variable domain, but also includes single variable domains from other species such as rodent nurse shark and Camelid V_{HH} dAbs™. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanized according to standard techniques available in the art, and such domains are considered to be “single variable domains”. As used herein V_{H} includes camelid V_{HH} domains.

An antigen binding fragment may be provided by means of arrangement of one or more CDRs on non-antibody protein scaffolds. “Protein Scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example an IgG, or IgA scaffold. The IgG scaffold may comprise some or all the domains of an antibody (i.e. CH1, CH2, CH3, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

The protein scaffold may be a derivative of a scaffold selected from the group consisting of CTLA-4, lipocalin, Protein A derived molecules such as Z-domain of Protein A (Affibody, SpA), A-domain (Avimer/Maxibody); heat shock proteins such as GroEl and GroES; transferrin (trans-body); ankyrin repeat protein (DARPin); peptide aptamer; C-type lectin domain (Tetranectin); human γ-crystallin and human ubiquitin (affilins); PDZ domains; scorpion toxin kunitz type domains of human protease inhibitors; and fibronectin/adnectin; which has been subjected to protein engineering in order to obtain binding to an antigen, such as IL1RAP, other than the natural ligand.

Antigen binding site refers to a site on an antigen binding protein which is capable of specifically binding to an antigen, this may be a single variable domain, or it may be paired V_{H}/V_{L} domains as can be found on a standard antibody. Single-chain Fv (ScFv) domains can also provide antigen-binding sites. The term “epitope-binding domain” refers to a domain that specifically binds to a region of an antigen known as the epitope independently of a different domain.

The term multi-specific antigen binding protein refers to antigen binding proteins which comprise at least two different antigen binding sites. Each of these antigen-binding sites will be capable of binding to a different epitope, which may be present on the same antigen or different antigens. The multi-specific antigen binding protein will have specificity for more than one antigen, for example two antigens, or for three antigens, or for four antigens.

Examples of multi-specific antigen binding proteins include those that consist of, or consist essentially of, an Fc region of an antibody, or a part thereof, linked at each end, directly or indirectly (for example, via a linker sequence) to a binding domain. Such an antigen binding protein may comprise two binding domains separated by an Fc region, or part thereof. By separated is meant that the binding domains are not directly linked to one another, and may be located at opposite ends (C and N terminus) of an Fc region, or any other scaffold region.

The antigen binding protein may comprise two scaffold regions each bound to two binding domains, for example at the N and C termini of each scaffold region, either directly or indirectly via a linker. Each binding domain may bind to a different antigen.

As used herein, the term mAbdAb refers to a monoclonal antibody linked to a further binding domain, in particular a single variable domain such as a domain antibody. A mAbdAb has at least two antigen binding sites, at least one of which is from a domain antibody, and at least one is from a paired V_{H}/V_{L} domain.

A “dAb™ conjugate” refers to a composition comprising a dAb to which a drug is chemically conjugated by means of a covalent or noncovalent linkage. Preferably, the dAb and the drug are covalently bonded. Such covalent linkage could be through a peptide bond or other means such as via a modified side chain. The noncovalent bonding may be direct (e.g., electrostatic interaction, hydrophobic interaction) or indirect (e.g., through noncovalent binding of complementary binding partners (e.g., biotin and avidin), wherein one partner is covalently bonded to drug and the complementary binding partner is covalently bonded to the dAb™). When complementary binding partners are employed, one of the binding partners can be covalently bonded to the drug directly or through a suitable linker moiety, and the complementary binding partner can be covalently bonded to the dAb™ directly or through a suitable linker moiety.

As used herein, “dAb™ fusion” refers to a fusion protein that comprises a dAb™ and a polypeptide drug (which could be a dAb™ or mAb). The dAb™ and the polypeptide drug are present as discrete parts (moieties) of a single continuous polypeptide chain.

In one embodiment, antigen binding proteins of the present disclosure may show cross-reactivity between human IL1RAP and IL1RAP from another species, such as cynomolgus IL1RAP. In an embodiment, the antigen binding proteins of the invention specifically bind human and cynomolgus IL1RAP. The provision of a drug that can bind human and monkey species allows one to test results in these systems and make side-by-side comparisons of data using the same drug. Cross reactivity between other species used in disease models such as dog or monkey, in particular monkey, is envisaged.

The term “neutralizes” as used throughout the present specification means that the interaction between IL1RAP and IL1RAP ligands (such as IL-1apha, IL-beta, IL-33, and IL-36 or receptors thereto) is reduced in the presence of an antigen binding protein as described herein in comparison to the interaction of IL1RAP and IL1RAP ligands in the absence of the IL1RAP binding protein, in vitro or in vivo. Neutralization may be due to one or more of blocking IL1RAP binding to its ligand, preventing IL1RAP from being activated by its ligand, down regulating IL1RAP or its receptor, or affecting effector functionality.

The effect of an IL1RAP binding protein on the interaction between IL1RAP and IL1RAP ligands may be partial or total. A neutralising IL1RAP binding protein may block the interaction of IL1RAP with IL1RAP-L (IL1RAP ligand) by at least 20%, 30% 40%, 50%, 55%, 60%, 65%, 70%, 75%, 80%, 82%, 84%, 86%, 88%, 90%, 92%, 94%, 95%, 96%, 97%, 98%, 99% or 100% relative to IL1RAP - IL1RAP ligand interactions in the absence of the IL1RAP binding protein.

Neutralization may be determined or measured using one or more assays known to the skilled person or as described herein.

Affinity is the strength of binding of one molecule, e.g. an antigen binding protein of the invention, to another, e.g. its target antigen, at a single binding site. The binding affinity of an antigen binding protein to its target may be determined by equilibrium methods (e.g. enzyme-linked immunoabsorbent assay (ELISA) or radioimmunoassay (RIA)), or kinetics (e.g. BIACORE™ analysis). For example, the Biacore™ methods described in Example 5 may be used to measure binding affinity.

Avidity is the sum total of the strength of binding of two molecules to one another at multiple sites, e.g., taking into account the valency of the interaction.

In an embodiment, the equilibrium dissociation constant (KD) of the IL1RAP binding protein-IL1RAP interaction is 100 nM or less, 10 nM or less, 2 nM or less or 1 nM or less. Alternatively, the KD may be between 5 and 10 nM; or between 1 and 2 nM. The KD may be between 1 pM and 500 pM; or between 500 pM and 1 nM. A skilled person will appreciate that the smaller the KD numerical value, the stronger the binding. The reciprocal of KD (i.e. 1/KD) is the equilibrium association constant (KA) having units M⁻¹. A skilled person will appreciate that the larger the KA numerical value, the stronger the binding.

The dissociation rate constant (kd) or “off-rate” describes the stability of the IL1RAP binding protein IL1RAP complex, i.e. the fraction of complexes that decay per second. For example, a kd of 0.01 s⁻¹ equates to 1% of the complexes decaying per second. In an embodiment, the dissociation rate constant (kd) is 1×10⁻³ s⁻¹ or less, 1×10⁻⁴ s⁻¹ or less, 1×10⁻⁵ s⁻¹ or less, or 1×10⁻⁶ s⁻¹ or less. The kd may be between 1×10⁻⁵ s⁻¹ and 1×10⁻⁴ s⁻¹; or between 1×10⁻⁴ s⁻¹ and 1×10⁻³ s⁻¹.

The association rate constant (ka) or “on-rate” describes the rate of IL1RAP binding protein-IL1RAP complex formation. In an embodiment, the association rate constant (ka) is about 1.0 × 10⁵ M⁻¹s⁻¹.

By “isolated” it is intended that the molecule, such as an antigen binding protein or nucleic acid, is removed from the environment in which it may be found in nature. For example, the molecule may be purified away from substances with which it would normally exist in nature. For example, the mass of the molecule in a sample may be 95% of the total mass.

The term “expression vector” as used herein means an isolated nucleic acid which can be used to introduce a nucleic acid of interest into a cell, such as a eukaryotic cell or prokaryotic cell, or a cell free expression system where the nucleic acid sequence of interest is expressed as a peptide chain such as a protein. Such expression vectors may be, for example, cosmids, plasmids, viral sequences, transposons, and linear nucleic acids comprising a nucleic acid of interest. Once the expression vector is introduced into a cell or cell free expression system (e.g., reticulocyte lysate) the protein encoded by the nucleic acid of interest is produced by the transcription/translation machinery. Expression vectors within the scope of the disclosure may provide necessary elements for eukaryotic or prokaryotic expression and include viral promoter driven vectors, such as CMV promoter driven vectors, e.g., pcDNA3.1, pCEP4, and their derivatives, Baculovirus expression vectors, Drosophila expression vectors, and expression vectors that are driven by mammalian gene promoters, such as human Ig gene promoters. Other examples include prokaryotic expression vectors, such as T7 promoter driven vectors, e.g., pET41, lactose promoter driven vectors and arabinose gene promoter driven vectors. Those of ordinary skill in the art will recognize many other suitable expression vectors and expression systems.

The term “recombinant host cell” as used herein means a cell that comprises a nucleic acid sequence of interest that was isolated prior to its introduction into the cell. For example, the nucleic acid sequence of interest may be in an expression vector while the cell may be prokaryotic or eukaryotic. Exemplary eukaryotic cells are mammalian cells, such as but not limited to, COS-1, COS-7, HEK293, BHK21, CHO, BSC-1, HepG2, 653, SP2/0, NS0, 293, HeLa, myeloma, lymphoma cells or any derivative thereof. Most preferably, the eukaryotic cell is a HEK293, NS0, SP2/0, or CHO cell. E. coli is an exemplary prokaryotic cell. A recombinant cell according to the disclosure may be generated by transfection, cell fusion, immortalization, or other procedures well known in the art. A nucleic acid sequence of interest, such as an expression vector, transfected into a cell may be extrachromasomal or stably integrated into the chromosome of the cell.

The IL1Rap binding proteins, for example antibodies of the present invention may be produced by transfection of a host cell with an expression vector comprising the coding sequence for the antigen binding protein of the invention. An expression vector or recombinant plasmid is produced by placing these coding sequences for the antigen binding protein in operative association with conventional regulatory control sequences capable of controlling the replication and expression in, and/or secretion from, a host cell. Regulatory sequences include promoter sequences, e.g., CMV promoter, and signal sequences which can be derived from other known antibodies. Similarly, a second expression vector can be produced having a DNA sequence which encodes a complementary antigen binding protein light or heavy chain. In certain embodiments this second expression vector is identical to the first except insofar as the coding sequences and selectable markers are concerned, so to ensure as far as possible that each polypeptide chain is functionally expressed. Alternatively, the heavy and light chain coding sequences for the antigen binding protein may reside on a single vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antigen binding protein of the invention. The antigen binding protein which includes the association of both the recombinant heavy chain and/or light chain is screened from culture by appropriate assay, such as ELISA or RIA. Similar conventional techniques may be employed to construct other antigen binding proteins.

Suitable vectors for the cloning and subcloning steps employed in the methods and construction of the compositions of this invention may be selected by one of skill in the art. For example, the conventional pUC series of cloning vectors may be used. One vector, pUC19, is commercially available from supply houses, such as Amersham (Buckinghamshire, United Kingdom) or Pharmacia (Uppsala, Sweden). Additionally, any vector which is capable of replicating readily, has an abundance of cloning sites and selectable genes (e.g., antibiotic resistance), and is easily manipulated may be used for cloning. Thus, the selection of the cloning vector is not a limiting factor in this invention.

The expression vectors may also be characterized by genes suitable for amplifying expression of the heterologous DNA sequences, e.g., the mammalian dihydrofolate reductase gene (DHFR). Other vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g., replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the antigen binding proteins of the present invention. Host cells useful for the cloning and other manipulations of these cloning vectors are also conventional. However, cells from various strains of E. Coli may be used for replication of the cloning vectors and other steps in the construction of antigen binding proteins of this invention.

Suitable host cells or cell lines for the expression of the antigen binding proteins of the invention include mammalian cells such as NS0, Sp2/0, CHO (e.g. DG44), COS, HEK, a fibroblast cell (e.g., 3T3), and myeloma cells, for example, it may be expressed in a CHO or a myeloma cell. Human cells may be used, thus enabling the molecule to be modified with human glycosylation patterns. Alternatively, other eukaryotic cell lines may be employed. The selection of suitable mammalian host cells and methods for transformation, culture, amplification, screening and product production and purification are known in the art. See, e.g., Sambrook et al., cited above.

Bacterial cells may prove useful as host cells suitable for the expression of the recombinant Fabs or other embodiments of the present invention (see, e.g., Plückthun, A., Immunol. Rev., 130:151-188 (1992)). However, due to the tendency of proteins expressed in bacterial cells to be in an unfolded or improperly folded form or in a non-glycosylated form, any recombinant Fab produced in a bacterial cell would have to be screened for retention of antigen binding ability. If the molecule expressed by the bacterial cell was produced in a properly folded form, that bacterial cell would be a desirable host, or in alternative embodiments the molecule may express in the bacterial host and then be subsequently re-folded. For example, various strains of E. Coli used for expression are well-known as host cells in the field of biotechnology. Various strains of B. Subtilis, Streptomyces, other bacilli and the like may also be employed in this method. Where desired, strains of yeast cells known to those skilled in the art are also available as host cells, as well as insect cells, e.g., Drosophila and Lepidoptera and viral expression systems. See, e.g., Miller et al., Genetic Engineering, 8:277-298, Plenum Press (1986) and references cited therein.

The general methods by which the vectors may be constructed, the transfection methods required to produce the host cells of the invention, and culture methods necessary to produce the antigen binding protein of the invention from such host cell may all be conventional techniques. Typically, the culture method of the present invention is a serum-free culture method, usually by culturing cells serum-free in suspension. Likewise, once produced, the antigen binding proteins of the invention may be purified from the cell culture contents according to standard procedures of the art, including ammonium sulfate precipitation, affinity columns, column chromatography, gel electrophoresis and the like. Such techniques are within the skill of the art and do not limit this invention. For example, preparations of altered antibodies are described in WO 99/58679 and WO 96/16990.

Yet another method of expression of the antigen binding proteins may utilize expression in a transgenic animal, such as described in U. S. Pat. No. 4,873,316. This relates to an expression system using the animals casein promoter which when transgenically incorporated into a mammal permits the female to produce the desired recombinant protein in its milk.

In a further embodiment of the invention, there is provided a method of producing an antibody of the invention, which method comprises the step of culturing a host cell transformed or transfected with a vector encoding the light and/or heavy chain of the antibody of the invention and recovering the antibody thereby produced.

In accordance with the present invention, there is provided a method of producing an anti-IL1 RAP antibody of the present invention which binds to and neutralises the activity of human IL1RAP, which method comprises the steps of;
providing a first vector encoding a heavy chain of the antibody;
providing a second vector encoding a light chain of the antibody;
transforming a mammalian host cell (e.g., CHO) with said first and second vectors;
culturing the host cell of step (c) under conditions conducive to the secretion of the antibody from said host cell into said culture media; and
recovering the secreted antibody of step (d).

In one embodiment of the present invention, there is provided a recombinant transformed, transfected or transduced host cell comprising at least one expression cassette, for example, where the expression cassette comprises a polynucleotide encoding a heavy chain of an antigen binding protein according to the invention described herein and further comprises a polynucleotide encoding a light chain of an antigen binding protein according to the invention described herein or where there are two expression cassettes and the 1st encodes the light chain and the second encodes the heavy chain. For example, in one embodiment the first expression cassette comprises a polynucleotide encoding a heavy chain of an antigen binding protein comprising a constant region or antigen binding fragment thereof which is linked to a constant region according to the invention described herein and further comprises a second cassette comprising a polynucleotide encoding a light chain of an antigen binding protein comprising a constant region or antigen binding fragment thereof which is linked to a constant region according to the invention described herein for example the first expression cassette comprises a polynucleotide encoding a heavy chain of SEQ ID NO:55, and a second expression cassette comprising a polynucleotide encoding a light chain of SEQ ID NO:56.

In another embodiment of the invention, there is provided a stably transformed host cell comprising a vector comprising one or more expression cassettes encoding a heavy chain and/or a light chain of the antibody comprising a constant region or antigen binding fragment thereof which is linked to a constant region as described herein. For example, such host cells may comprise a first vector encoding the light chain and a second vector encoding the heavy chain, for example, the first vector encodes a heavy chain of SEQ ID NO:55, and a second vector encoding a light chain of SEQ ID NO:56.

In another embodiment of the present invention there is provided a host cell according to the invention described herein wherein the cell is eukaryotic, for example where the cell is mammalian. Examples of such cell lines include CHO or NS0.

In another embodiment of the present invention, there is provided a method for the production of an antibody comprising a constant region or antigen binding fragment thereof which is linked to a constant region according to the invention described herein which method comprises the step of culturing a host cell in a culture media, for example, serum-free culture media.

In another embodiment of the present invention, there is provided a method according to the invention described herein, wherein said antibody is further purified to at least 95% or greater (e.g., 98% or greater) with respect to said antibody containing serum-free culture media.

In yet another embodiment, there is provided a pharmaceutical composition comprising an antigen binding protein and a pharmaceutically acceptable carrier.

In another embodiment of the present invention, there is provided a kit-of-parts comprising the composition according to the invention described herein, described together with instructions for use.

The mode of administration of the therapeutic agent of the invention may be any suitable route which delivers the agent to the host. The antigen binding proteins and pharmaceutical compositions of the invention are particularly useful for parenteral administration, i.e., subcutaneously (s.c.), intrathecally, intraperitoneally, intramuscularly (i.m.) or intravenously (i.v.). In one such embodiment, the antigen binding proteins of the present invention are administered intravenously or subcutaneously.

Therapeutic agents of the invention may be prepared as pharmaceutical compositions containing an effective amount of the antigen binding protein of the invention as an active ingredient in a pharmaceutically acceptable carrier. In one embodiment, the prophylactic agent of the invention is an aqueous suspension or solution containing the antigen binding protein in a form ready for injection. In one embodiment, the suspension or solution is buffered at physiological pH. In one embodiment, the compositions for parenteral administration will comprise a solution of the antigen binding protein of the invention or a cocktail thereof dissolved in a pharmaceutically acceptable carrier. In one embodiment, the carrier is an aqueous carrier. A variety of aqueous carriers may be employed, e.g., 0.9% saline, 0.3% glycine, and the like. These solutions may be made sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antigen binding protein of the invention in such pharmaceutical formulation can vary widely, i.e., from less than about 0.5%, usually at or at least about 1% to as much as about 15 or 20% by weight and will be selected primarily based on fluid volumes, viscosities, etc., according to the particular mode of administration selected.

Thus, a pharmaceutical composition of the invention for intravenous infusion could be made up to contain about 250 ml of sterile Ringer’s solution, and about 1 to about 30 or 5 mg to about 25 mg of an antigen binding protein of the invention per ml of Ringer’s solution. Actual methods for preparing parenterally administrable compositions are well known or will be apparent to those skilled in the art and are described in more detail in, for example, Remington’s Pharmaceutical Science, 15th ed., Mack Publishing Company, Easton, Pennsylvania. For the preparation of intravenously administrable antigen binding protein formulations of the invention, see Lasmar U and Parkins D “The formulation of Biopharmaceutical products”, Pharma. Sci.Tech.today, page 129-137, Vol. 3 (3rd April 2000); Wang, W “Instability, stabilisation and formulation of liquid protein pharmaceuticals”, Int. J. Pharm 185 (1999) 129-188; Stability of Protein Pharmaceuticals Part A and B ed Ahern T.J., Manning M.C., New York, NY: Plenum Press (1992); Akers, M.J. “Excipient-Drug interactions in Parenteral Formulations”, J Pharm Sci 91 (2002) 2283-2300; Imamura, K et al “Effects of types of sugar on stabilization of Protein in the dried state”, J Pharm Sci 92 (2003) 266-274; Izutsu, Kkojima, S. “Excipient crystallinity and its protein-structure-stabilizing effect during freeze-drying”, J Pharm. Pharmacol, 54 (2002) 1033-1039; Johnson, R, “Mannitol-sucrose mixtures-versatile formulations for protein lyophilization”, J. Pharm. Sci, 91 (2002) 914-922; and Ha, E Wang W, Wang Y.J. “Peroxide formation in polysorbate 80 and protein stability”, J. Pharm Sci, 91, 2252-2264,(2002) the entire contents of which are incorporated herein by reference and to which the reader is specifically referred.

In one embodiment the therapeutic agent of the invention, when in a pharmaceutical preparation, is present in unit dose forms. The appropriate therapeutically effective dose will be determined readily by those of skill in the art. Suitable doses may be calculated for patients according to their weight, for example suitable doses may be in the range of about 0.1 to about 20 mg/kg, for example about 1 to about 20 mg/kg, for example about 10 to about 20 mg/kg or for example about 1 to about 15 mg/kg, for example about 10 to about 15 mg/kg. Preferably, an IL1RAP mAb could be administered as an intravenous infusion dose of 1 mg/kg.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one or more human immunoglobulin(s). In addition, framework support residues may be altered to preserve binding affinity (see, e.g., Queen et al. Proc. Natl Acad Sci USA, 86:10029-10032 (1989), Hodgson, et al., Bio/Technology, 9:421 (1991)). A suitable human acceptor antibody may be one selected from a conventional database, e.g., the KABAT™ database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies - see, for example, EP-A-0239400 and EP-A-054951.

The term “fully human antibody” includes antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). Fully human antibodies comprise amino acid sequences encoded only by polynucleotides that are ultimately of human origin or amino acid sequences that are identical to such sequences. As meant herein, antibodies encoded by human immunoglobulin-encoding DNA inserted into a mouse genome produced in a transgenic mouse are fully human antibodies since they are encoded by DNA that is ultimately of human origin. In this situation, human immunoglobulin-encoding DNA can be rearranged (to encode an antibody) within the mouse, and somatic mutations may also occur. Antibodies encoded by originally human DNA that has undergone such changes in a mouse are fully human antibodies as meant herein. The use of such transgenic mice makes it possible to select fully human antibodies against a human antigen. As is understood in the art, fully human antibodies can be made using phage display technology wherein a human DNA library is inserted in phage for generation of antibodies comprising human germline DNA sequence.

The term “donor antibody” refers to an antibody that contributes the amino acid sequences of its variable regions, CDRs, or other functional fragments or analogs thereof to a first immunoglobulin partner. The donor, therefore, provides the altered immunoglobulin coding region and resulting expressed altered antibody with the antigenic specificity and neutralising activity characteristic of the donor antibody.

The term “acceptor antibody” refers to an antibody that is heterologous to the donor antibody, which contributes all (or any portion) of the amino acid sequences encoding its heavy and/or light chain framework regions and/or its heavy and/or light chain constant regions to the first immunoglobulin partner. A human antibody may be the acceptor antibody.

The terms “V_{H}” and “V_{L}” are used herein to refer to the heavy chain variable region and light chain variable region respectively of an antigen binding protein.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 5th Ed., U.S. Department of Health and Human Services, National Institutes of Health (1991).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 1

| | Kabat CDR | Chothia CDR | AbM CDR | Contact CDR | Minimum binding unit |
|---|---|---|---|---|---|
| H1 | 31-35/35A/ 35B | 26-32/33/34 | 26-35/35A/35B | 30-35/35A/35B | 31-32 |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

CDRs or minimum binding units may be modified by at least one amino acid substitution, deletion or addition, wherein the variant antigen binding protein substantially retains the biological characteristics of the unmodified protein.

It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution, for example as shown in Table 2 below.

TABLE 2

| | |
|---|---|
| Side chain | Members |
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

For example, in a variant CDR, the amino acid residues of the minimum binding unit may remain the same, but the flanking residues that comprise the CDR as part of the Kabat or Chothia definition(s) may be substituted with a conservative amino acid residue.

Such antigen binding proteins comprising modified CDRs or minimum binding units as described above may be referred to herein as “functional CDR variants” or “functional binding unit variants”. Suitably, in one embodiment, IL1RAP binding proteins are provided comprising one or more CDRs having the amino acid sequences set forth in SEQ ID NOs:33, 34, 35, 38, 39, and/or 40 and/or a functional CDR variant thereof.

The term “epitope” as used herein refers to that portion of the antigen that makes contact with a particular binding domain of the antigen binding protein. An epitope may be linear or conformational/discontinuous. A conformational or discontinuous epitope comprises amino acid residues that are separated by other sequences, i.e., not in a continuous sequence in the antigen’s primary sequence. Although the residues may be from different regions of the peptide chain, they are in close proximity in the three dimensional structure of the antigen. In the case of multimeric antigens, a conformational or discontinuous epitope may include residues from different peptide chains. Particular residues comprised within an epitope can be determined through computer modelling programs or via three-dimensional structures obtained through methods known in the art, such as X-ray crystallography.

The CDRs L1, L2, L3, H1 and H2 tend to structurally exhibit one of a finite number of main chain conformations. The particular canonical structure class of a CDR is defined by both the length of the CDR and by the loop packing, determined by residues located at key positions in both the CDRs and the framework regions (structurally determining residues or SDRs). Martin and Thornton (1996; J Mol Biol 263:800-815) have generated an automatic method to define the “key residue” canonical templates. Cluster analysis is used to define the canonical classes for sets of CDRs, and canonical templates are then identified by analysing buried hydrophobics, hydrogen-bonding residues, and conserved glycines and prolines. The CDRs of antibody sequences can be assigned to canonical classes by comparing the sequences to the key residue templates and scoring each template using identity or similarity matrices.

There may be multiple variant CDR canonical positions per CDR, per corresponding CDR, per binding unit, per heavy or light chain variable region, per heavy or light chain, and per antigen binding protein, and therefore any combination of substitution may be present in the antigen binding protein of the invention, provided that the canonical structure of the CDR is maintained such that the antigen binding protein is capable of specifically binding IL1RAP.

As discussed above, the particular canonical structure class of a CDR is defined by both the length of the CDR and by the loop packing, determined by residues located at key positions in both the CDRs and the framework regions.

“Percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute’s website with the filter for low complexity regions turned off. Importantly, a query nucleic acid sequence may be described by a nucleic acid sequence identified in one or more claims herein.

“Percent identity” between a query amino acid sequence and a subject amino acid sequence is the “Identities” value, expressed as a percentage, that is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute’s website with the filter for low complexity regions turned off. Importantly, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The % identity may be determined across the entire length of the query sequence, including the CDR(s). Alternatively, the % identity may exclude the CDR(s), for example the CDR(s) is 100% identical to the subject sequence and the % identity variation is in the remaining portion of the query sequence, so that the CDR sequence is fixed/intact.

In certain embodiments:
(1) Identity for polynucleotides is calculated by multiplying the total number of nucleotides in a given sequence by the integer defining the percent identity divided by 100 and then subtracting that product from said total number of nucleotides in said sequence, or:

$$\text{nn} \leq \text{xn} - \left(\text{xn} \cdot \text{y}\right) ,$$
wherein nn is the number of nucleotide alterations, xn is the total number of nucleotides in a given sequence, y is 0.95 for 95%, 0.97 for 97% or 1.00 for 100%, and ● is the symbol for the multiplication operator, and wherein any non-integer product of xn and y is rounded down to the nearest integer prior to subtracting it from xn. Alterations of a polynucleotide sequence encoding a polypeptide may create nonsense, missense or frameshift mutations in this coding sequence and thereby alter the polypeptide encoded by the polynucleotide following such alterations.
(2) Identity for polypeptides is calculated by multiplying the total number of amino acids by the integer defining the percent identity divided by 100 and then subtracting that product from said total number of amino acids, or:
   na ≤ xa - (xa ● y), wherein na is the number of amino acid alterations, xa is the total number of amino acids in the sequence, y is 0.95 for 95%, 0.97 for 97% or 1.00 for 100%, and ● is the symbol for the multiplication operator, and wherein any non-integer product of xa and y is rounded down to the nearest integer prior to subtracting it from xa.

The variant sequence substantially retains the biological characteristics of the unmodified protein, such as SEQ ID NO:51, 52, 55, or 56.

The V_{H} or V_{L} sequence may be a variant sequence with up to 15 amino acid substitutions, additions or deletions. For example, the variant sequence may have up to 15, 14, 13, 12, 11, 10, 9, 8, 7, 6, 5, 4, 3, 2 or 1 amino acid substitution(s), addition(s) or deletion(s).

The sequence variation may exclude the CDR(s), for example the CDR(s) is the same as the V_{H} or V_{L} sequence and the variation is in the remaining portion of the V_{H} or V_{L} sequence, so that the CDR sequence is fixed/intact.

The skilled person will appreciate that, upon production of an antigen binding protein such as an antibody, in particular depending on the cell line used and particular amino acid sequence of the antigen binding protein, post-translational modifications may occur. For example, this may include the cleavage of certain leader sequences, the addition of various sugar moieties in various glycosylation and phosphorylation patterns, deamidation, oxidation, disulfide bond scrambling, isomerisation, C-terminal lysine clipping, and N-terminal glutamine cyclisation. The present invention encompasses the use of antigen binding proteins which have been subjected to, or have undergone, one or more post-translational modifications. Thus an “antigen binding protein” or “antibody” of the invention includes an “antigen binding protein” or “antibody”, respectively, as defined earlier which has undergone a post-translational modification such as described herein.

Deamidation is an enzymatic reaction primarily converting asparagine (N) to iso-aspartic acid and aspartic acid (D) at approximately 3:1 ratio. To a much lesser degree, deamidation can occur with glutamine residues in a similar manner. Deamidation in a CDR results in a change in charge of the molecule, but typically does not result in a change in antigen binding, nor does it impact on PK/PD.

Oxidation can occur during production and storage (i.e. in the presence of oxidizing conditions) and results in a covalent modification of a protein, induced either directly by reactive oxygen species or indirectly by reaction with secondary by-products of oxidative stress. Oxidation happens primarily with methionine residues, but occasionally can occur at tryptophan and free cysteine residues.

Disulfide bond scrambling can occur during production and basic storage conditions. Under certain circumstances, disulfide bonds can break or form incorrectly, resulting in unpaired cysteine residues (—SH). These free (unpaired) sulfhydryls (—SH) can promote shuffling.

Isomerization typically occurs during production, purification, and storage (at acidic pH) and usually occurs when aspartic acid is converted to isoaspartic acid through a chemical process.

N-terminal glutamine in the heavy chain and/or light chain is likely to form pyroglutamate (pGlu). Most pGlu formation happens in the production bioreactor, but it can be formed non-enzymatically, depending on pH and temperature of processing and storage conditions. pGlu formation is considered as one of the principal degradation pathways for recombinant mAbs.

C-terminal lysine clipping is an enzymatic reaction catalyzed by carboxypeptidases, and is commonly observed in recombinant mAbs. Variants of this process include removal of lysine from one or both heavy chains. Lysine clipping does not appear to impact bioactivity and has no effect on mAb effector function.

Naturally occurring autoantibodies exist in humans that can bind to proteins. Autoantibodies can thus bind to endogenous proteins (present in naive subjects) as well as to proteins or peptides which are administered to a subject for treatment. Therapeutic protein-binding autoantibodies and antibodies that are newly formed in response to drug treatment are collectively termed anti-drug antibodies (ADAs). Pre-existing antibodies against molecules such as therapeutic proteins and peptides, administered to a subject can affect their efficacy and could result in administration reactions, hypersensitivity, altered clinical response in treated patients and altered bioavailability by sustaining, eliminating or neutralizing the molecule. It could be advantageous to provide molecules for therapy which comprise human immunoglobulin (antibody) single variable domains or dAbs™ which have reduced immunogenicity (i.e., reduced ability to bind to pre-existing ADAs when administered to a subject, in particular a human subject.

Thus, in one embodiment of the present invention there is provided a modified dAb™ which has reduced ability to bind to pre-existing antibodies (ADAs) as compared to the equivalent unmodified molecule. By reduced ability to bind it is meant that the modified molecule binds with a reduced affinity or reduced avidity to a pre-existing ADA. Said modified dAb™ comprise one or more modifications selected from: (a) a C-terminal addition, extension, deletion or tag, and/or (b) one or more amino acid framework substitutions.

Polypeptides and dAbs™ of the disclosure and agonists comprising these can be formatted to have a larger hydrodynamic size, for example, by attachment of a PEG group, serum albumin, transferrin, transferrin receptor or at least the transferrin-binding portion thereof, an antibody Fc region, or by conjugation to an antibody domain. For example, polypeptides dAbs™ and agonists may be formatted as a larger antigen-binding fragment of an antibody or as an antibody (e.g., formatted as a Fab, Fab′, F(ab)₂, F(ab′)₂, IgG, scFv).

As used herein, “hydrodynamic size” refers to the apparent size of a molecule (e.g., an antigen binding protein) based on the diffusion of the molecule through an aqueous solution. The diffusion or motion of a protein through solution can be processed to derive an apparent size of the protein, where the size is given by the “Stokes radius” or “hydrodynamic radius” of the protein particle. The “hydrodynamic size” of a protein depends on both mass and shape (conformation), such that two proteins having the same molecular mass may have differing hydrodynamic sizes based on the overall conformation and charge of the protein. An increase in hydrodynamic size can give an associated decrease in renal clearance leading to an observed increase in half-life (t_{½}).

Hydrodynamic size of the antigen binding proteins (e.g., domain antibody monomers and multimers) of the disclosure may be determined using methods which are well known in the art. For example, gel filtration chromatography may be used to determine the hydrodynamic size of an antigen binding protein. Suitable gel filtration matrices for determining the hydrodynamic sizes of antigen binding proteins, such as cross-linked agarose matrices, are well known and readily available.

The size of an antigen binding protein format (e.g., the size of a PEG moiety attached to a domain antibody monomer), can be varied depending on the desired application. For example, where antigen binding protein is intended to leave the circulation and enter into peripheral tissues, it is desirable to keep the hydrodynamic size of the IL1RAP binding protein low to facilitate extravazation from the blood stream. Alternatively, where it is desired to have the antigen binding protein remain in the systemic circulation for a longer period of time the size of the antigen binding protein can be increased, for example by formatting as an Ig like protein.

### Further Description of Pharmaceutical Compositions

IL1RAP binding protein (or sometime referred to as antigen binding protein herein) as described herein may be incorporated into pharmaceutical compositions for use in the treatment of the human diseases described herein. In one embodiment, the pharmaceutical composition comprises an antigen binding protein optionally in combination with one or more pharmaceutically acceptable carriers and/or excipients.

Such compositions comprise a pharmaceutically acceptable carrier as known and called for by acceptable pharmaceutical practice.

Pharmaceutical compositions may be administered by injection or continuous infusion (examples include, but are not limited to, intravenous, intraperitoneal, intradermal, subcutaneous, intramuscular and intraportal). In one embodiment, the composition is suitable for intravenous administration. Pharmaceutical compositions may be suitable for topical administration (which includes, but is not limited to, epicutaneous, inhaled, intranasal or ocular administration) or enteral administration (which includes, but is not limited to, oral or rectal administration).

Pharmaceutical compositions may comprise between 0.5 mg to 10 g of IL1RAP binding protein, for example between 5 mg and 1 g of antigen binding protein. Alternatively, the composition may comprise between 5 mg and 500 mg, for example between 5 mg and 50 mg. Methods for the preparation of such pharmaceutical compositions are well known to those skilled in the art. Other excipients may be added to the composition as appropriate for the mode of administration and the particular protein used. Examples of different excipients and their uses are described in Lowe et al., (2011).

Effective doses and treatment regimes for administering the antigen binding protein may be dependent on factors such as the age, weight and health status of the patient and disease to be treated. Such factors are within the purview of the attending physician. Guidance in selecting appropriate doses may be found in e.g., Bai et al. (2012).

The pharmaceutical composition may comprise a kit of parts of the antigen binding protein together with other medicaments, optionally with instructions for use. For convenience, the kit may comprise the reagents in predetermined amounts with instructions for use.

The terms “individual”, “subject” and “patient” are used herein interchangeably. In one embodiment, the subject is a mammal, such as a primate, for example a marmoset or monkey. In another embodiment, the subject is a human.

The antigen binding protein described herein may also be used in methods of treatment. Treatment can be therapeutic, prophylactic or preventative. Treatment encompasses alleviation, reduction, or prevention of at least one aspect or symptom of a disease and encompasses prevention or cure of the diseases described herein.

The antigen binding protein described herein is used in an effective amount for therapeutic, prophylactic or preventative treatment. A therapeutically effective amount of the antigen binding protein described herein is an amount effective to ameliorate or reduce one or more symptoms of, or to prevent or cure, the disease.

Thus, in one embodiment, IL1RAP binding proteins of the present invention are provided for use in therapy. In one embodiment, IL1RAP binding proteins of the present invention are provided for use in the treatment of cancer or a disease associated with inflammatory cytokines. The present invention also includes the use of an IL1RAP binding protein of the present invention in the manufacture of a medicament for the treatment of cancer or a disease associated with inflammatory cytokine.

### Further Description of Production Methods

Antigen binding proteins may be prepared by any of a number of conventional techniques. For example, antigen binding proteins may purified from cells that naturally express them (e.g., an antibody can be purified from a hybridoma that produces it), or produced in recombinant expression systems.

A number of different expression systems and purification regimes can be used to generate the antigen binding protein of the invention. Generally, host cells are transformed with a recombinant expression vector encoding the desired antigen binding protein. A wide range of host cells can be employed, including Prokaryotes (including Gram negative or Gram positive bacteria, for example Escherichia coli, Bacilli sp., Pseudomonas sp., Corynebacterium sp.), Eukaryotes including yeast (for example Saccharomyces cerevisiae, Pichia pastoris), fungi (for example Aspergilus sp.), or higher Eukaryotes including insect cells and cell lines of mammalian origin (for example, CHO, Perc6, HEK293, HeLa).

The host cell may be an isolated host cell. The host cell is usually not part of a multicellular organism (e.g., plant or animal). The host cell may be a non-human host cell.

Appropriate cloning and expression vectors for use with bacterial, fungal, yeast, and mammalian cellular hosts and methods of cloning are known in the art.

The cells can be cultured under conditions that promote expression of the antigen binding protein, and the polypeptide recovered by conventional protein purification procedures. The antigen binding proteins contemplated for use herein include substantially homogeneous antigen binding proteins substantially free of contaminating materials.

The skilled person will appreciate that, upon production of the antigen binding protein, in particular depending on the cell line used and particular amino acid sequence of the antigen binding protein, post-translational modifications may occur. This may include the cleavage of certain leader sequences, the addition of various sugar moieties in various glycosylation patterns, deamidation (for example at an asparagine or glutamine residue), oxidation (for example at a methionine, tryptophan or free cysteine residue), disulfide bond scrambling, isomerisation (for example at an aspartic acid residue), C-terminal lysine clipping (for example from one or both heavy chains), and N-terminal glutamine cyclisation (for example in the heavy and/or light chain). The present invention encompasses the use of antibodies which have been subjected to, or have undergone, one or more post-translational modifications. The modification may occur in a CDR, the variable framework region, or the constant region. The modification may result in a change in charge of the molecule. The modification typically does not result in a change in antigen binding, function, bioactivity, nor does it impact the pharmacokinetic (PK) or pharmacodynamic (PD) characteristics of the IL1RAP binding protein.

The term “Effector Function” as used herein is meant to refer to one or more of Antibody dependant cell mediated cytotoxic activity (ADCC), Complement-dependant cytotoxic activity (CDC) mediated responses, Fc-mediated phagocytosis or antibody dependant cellular phagocytosis (ADCP) and antibody recycling via the FcRn receptor.

The interaction between the constant region of an antigen binding protein and various Fc receptors (FcR) including FcyRI (CD64), FcyRII (CD32) and FcyRIII (CD16) is believed to mediate the effector functions of the antigen binding protein. Significant biological effects can be a consequence of effector functionality. Usually, the ability to mediate effector function requires binding of the antigen binding protein to an antigen and not all antigen binding proteins will mediate every effector function.

Effector function can be measured in a number of ways including, for example, via binding of the FcyRIII on Natural Killer cells or via FcyRI on monocytes/macrophages to measure for ADCC/ADCP effector function. For example, an antigen binding protein of the present invention can be assessed for ADCC effector function in a Natural Killer cell assay. Practical approaches to evaluate ADCC and /or CDC function can be found in (Kellner C et al., “Boosting ADCC and CDC activity by Fc engineering and evaluation of antibody effector functions”, Methods, 1;65(1):105-13 (2014)).

Some isotypes of human constant regions, in particular IgG4 and IgG2 isotypes, have reduced function of a) activation of complement by the classical pathway; and b) antibody-dependent cellular cytotoxicity. Various modifications to the heavy chain constant region of antigen binding proteins may be carried out depending on the desired effector property. IgG1 constant regions containing specific mutations have separately been described to reduce binding to Fc receptors and therefore reduce ADCC and CDC. (Kellner C et al., “Boosting ADCC and CDC activity by Fc engineering and evaluation of antibody effector functions”, Methods, 1;65(1):105-13 (2014)).

In one embodiment of the present invention there is provided an antigen binding protein comprising a constant region such that the antigen binding protein has reduced ADCC and/or complement activation or effector functionality. In one such embodiment, the heavy chain constant region may comprise a naturally disabled constant region of IgG2 or IgG4 isotype or a mutated IgG1 constant region. One example comprises the substitutions of alanine residues at positions 235 and 237 (EU index numbering).

The subclass of an antibody in part determines secondary effector functions, such as complement activation or Fc receptor (FcR) binding and antibody dependent cell cytotoxicity (ADCC) (Huber, et al., Nature 229(5284): 419-20 (1971); Brunhouse, et al., Mol Immunol 16(11): 907-17 (1979)). In identifying the optimal type of antibody for a particular application, the effector functions of the antibodies can be taken into account. For example, hIgG1 antibodies have a relatively long half-life, are very effective at fixing complement, and they bind to both FcyRI and FcyRII. In contrast, human IgG4 antibodies have a shorter half-life, do not fix complement and have a lower affinity for the FcRs. Replacement of serine 228 with a proline (S228P) in the Fc region of IgG4 reduces heterogeneity observed with hIgG4 and extends the serum half-life (Kabat, et al., “Sequences of proteins of immunological interest” 5.sup.th Edition (1991); Angal, et al., Mol Immunol 30(1): 105-8 (1993)). A second mutation that replaces leucine 235 with a glutamic acid (L235E) eliminates the residual FcR binding and complement binding activities (Alegre, et al., J Immunol 148(11): 3461-8 (1992)). The resulting antibody with both mutations is referred to as IgG4PE. The numbering of the hIgG4 amino acids was derived from EU numbering reference: Edelman, G.M. et al., Proc. Natl. Acad. USA, 63, 78-85 (1969). PMID: 5257969. In one embodiment of the present invention IL1RAP antigen binding proteins comprising an IgG4 Fc region comprising the replacement S228P and L235E may have the designation IgG4PE.

### Enhanced ADCC/CDC

As is understood in the art, various techniques are known which will increase the ADCC and/or the CDC activity of an antibody. These include, but are not limited to, various mutation in the Fc region, COMPLEGENT™ and POTELLIGENT™ technologies. In one aspect of the present invention, one or more ADCC/CDC enhancing techniques may be applied to the IL1RAP binding proteins (in particular anti-IL1RAP antibodies) of the present invention.

### Mutation

Human IgG1 constant regions containing specific mutations or altered glycosylation on residue Asn297 have also been described to enhance binding to Fc receptors. In some cases these mutations have also been shown to enhance ADCC and CDC, see for example, Kellner (2013).

In one embodiment of the present invention, such mutations are in one or more of positions selected from 239, 332 and 330 (IgG1), or the equivalent positions in other IgG isotypes. Examples of suitable mutations are S239D and I332E and A330L. In one embodiment the antigen binding protein of the invention herein described is mutated at positions 239 and 332, for example S239D and I332E or in a further embodiment it is mutated at three or more positions selected from 239 and 332 and 330, for example S239D and I332E and A330L (EU index numbering).

### Complegent™

In one embodiment of the present invention, there is provided an antigen binding protein comprising a chimeric heavy chain constant region for example an antigen binding protein comprising a chimeric heavy chain constant region with at least one CH2 domain from IgG3 such that the antigen binding protein has enhanced effector function, for example wherein it has enhanced ADCC or enhanced CDC, or enhanced ADCC and CDC functions. In one such embodiment, the antigen binding protein may comprise one CH2 domain from IgG3 or both CH2 domains may be from IgG3.

Also provided is a method of producing an antigen binding protein according to the invention comprising the steps of:
a) culturing a recombinant host cell comprising an expression vector comprising an isolated nucleic acid as described herein wherein the expression vector comprises a nucleic acid sequence encoding an Fc domain having both IgG1 and IgG3 Fc domain amino acid residues; and
b) recovering the antigen binding protein.

Such methods for the production of antigen binding proteins can be performed, for example, using the COMPLEGENT™ technology system available from BioWa, Inc. (Princeton, NJ) and Kyowa Hakko Kogyo (now, Kyowa Hakko Kirin Co., Ltd.) Co., Ltd. In which a recombinant host cell comprising an expression vector in which a nucleic acid sequence encoding a chimeric Fc domain having both IgG1 and IgG3 Fc domain amino acid residues is expressed to produce an antigen binding protein having enhanced complement dependent cytotoxicity (CDC) activity that is increased relative to an otherwise identical antigen binding protein lacking such a chimeric Fc domain. Aspects of the COMPLEGENT™ technology system are described in WO2007011041 and US20070148165 each of which are incorporated herein by reference. In an alternative embodiment, CDC activity may be increased by introducing sequence specific mutations into the Fc region of an IgG chain. Those of ordinary skill in the art will also recognize other appropriate systems.

### Potelligent™

The present invention also provides a method for the production of an antigen binding protein according to the invention comprising the steps of:
a) culturing a recombinant host cell comprising an expression vector comprising the isolated nucleic acid as described herein, wherein the FUT8 gene encoding alpha-1,6-fucosyltransferase has been inactivated in the recombinant host cell; and
b) recovering the antigen binding protein.

Such methods for the production of antigen binding proteins can be performed, for example, using the POTELLIGENT™ technology system available from BioWa, Inc. (Princeton, NJ) in which CHOK1SV cells lacking a functional copy of the FUT8 gene produce monoclonal antibodies having enhanced antibody dependent cell mediated cytotoxicity (ADCC) activity that is increased relative to an identical monoclonal antibody produced in a cell with a functional FUT8 gene. Aspects of the POTELLIGENT™ technology system are described in US7214775, US6946292, WO0061739 and WO0231240 all of which are incorporated herein by reference. Those of ordinary skill in the art will also recognize other appropriate systems.

It will be apparent to those skilled in the art that such modifications may not only be used alone but may be used in combination with each other in order to further enhance effector function.

In one such embodiment of the present invention there is provided an antigen binding protein comprising a heavy chain constant region which comprises a mutated and chimeric heavy chain constant region for example wherein an antigen binding protein comprising at least one CH2 domain from IgG3 and one CH2 domain from IgG1, wherein the IgG1 CH2 domain has one or more mutations at positions selected from 239 and 332 and 330 (for example the mutations may be selected from S239D and I332E and A330L) such that the antigen binding protein has enhanced effector function, for example wherein it has one or more of the following functions, enhanced ADCC or enhanced CDC, for example wherein it has enhanced ADCC and enhanced CDC. In one embodiment, the IgG1 CH2 domain has the mutations S239D and I332E.

In an alternative embodiment of the present invention there is provided an antigen binding protein comprising a chimeric heavy chain constant region and which has an altered glycosylation profile. In one such embodiment the heavy chain constant region comprises at least one CH2 domain from IgG3 and one CH2 domain from IgG1 and has an altered glycosylation profile such that the ratio of fucose to mannose is 0.8:3 or less, for example wherein the antigen binding protein is defucosylated so that said antigen binding protein has an enhanced effector function in comparison with an equivalent antigen binding protein with an immunoglobulin heavy chain constant region lacking said mutations and altered glycosylation profile, for example wherein it has one or more of the following functions, enhanced ADCC or enhanced CDC, for example wherein it has enhanced ADCC and enhanced CDC.

In an alternative embodiment the antigen binding protein has at least one IgG3 CH2 domain and at least one heavy chain constant domain from IgG1 wherein both IgG CH2 domains are mutated in accordance with the limitations described herein.

In one aspect of the invention there is provided a method of producing an antigen binding protein according to the invention described herein comprising the steps of:
a) culturing a recombinant host cell containing an expression vector containing an isolated nucleic acid as described herein, said expression vector further comprising a Fc nucleic acid sequence encoding a chimeric Fc domain having both IgG1 and IgG3 Fc domain amino acid residues, and wherein the FUT8 gene encoding alpha-1,6-fucosyltransferase has been inactivated in the recombinant host cell; and
b) recovering the antigen binding protein.

Such methods for the production of antigen binding proteins can be performed, for example, using the ACCRETAMAB™ technology system available from BioWa, Inc. (Princeton, NJ) which combines the POTELLIGENT™ and COMPLEGENT™ technology systems to produce an antigen binding protein having both ADCC and CDC enhanced activity that is increased relative to an otherwise identical monoclonal antibody lacking a chimeric Fc domain and which has fucose on the oligosaccharide.

In yet another embodiment of the present invention there is provided an antigen binding protein comprising a mutated and chimeric heavy chain constant region wherein said antigen binding protein has an altered glycosylation profile such that the antigen binding protein has enhanced effector function, for example wherein it has one or more of the following functions, enhanced ADCC or enhanced CDC. In one embodiment the mutations are selected from positions 239 and 332 and 330, for example the mutations are selected from S239D and I332E and A330L. In a further embodiment the heavy chain constant region comprises at least one CH2 domain from IgG3 and one Ch2 domain from IgG1. In one embodiment the heavy chain constant region has an altered glycosylation profile such that the ratio of fucose to mannose is 0.8:3 or less for example the antigen binding protein is defucosylated, so that said antigen binding protein has an enhanced effector function in comparison with an equivalent non-chimeric antigen binding protein or with an immunoglobulin heavy chain constant region lacking said mutations and altered glycosylation profile.

The long half-life of IgG antibodies is reported to be dependent on its binding to FcRn. Therefore, substitutions that increase the binding affinity of IgG to FcRn at pH 6.0 while maintaining the pH dependence of the interaction by engineering the constant region have been extensively studied Kuo and Aveson (2011).

Another means of modifying antigen binding proteins of the present invention involves increasing the in-vivo half-life of such proteins by modification of the immunoglobulin constant domain or FcRn (Fc receptor neonate) binding domain.

In adult mammals, FcRn, also known as the neonatal Fc receptor, plays a key role in maintaining serum antibody levels by acting as a protective receptor that binds and salvages antibodies of the IgG isotype from degradation. IgG molecules are endocytosed by endothelial cells, and if they bind to FcRn, are recycled out into circulation. In contrast, IgG molecules that do not bind to FcRn enter the cells and are targeted to the lysosomal pathway where they are degraded.

The neonatal FcRn receptor is believed to be involved in both antibody clearance and the transcytosis across tissues, Kuo and Aveson, (2011). Human IgG1 residues determined to interact directly with human FcRn includes Ile253, Ser254, Lys288, Thr307, Gln311, Asn434 and His435. Switches at any of these positions described in this section may enable increased serum half-life and/or altered effector properties of antigen binding proteins of the invention.

### Mutations to Increase Half-Life by Increasing Affinity to FcRn

Antigen binding proteins of the present invention may have one or more amino acid modifications that increase the affinity of the constant domain or fragment thereof for FcRn. These may result in increased half-life of these proteins Kuo and Aveson (2011). Increasing the half-life of therapeutic and diagnostic IgG’s and other bioactive molecules has many benefits including reducing the amount and/or frequency of dosing of these molecules. In one embodiment, there is therefore provided an antigen binding according to the invention provided herein or a fusion protein comprising all or a portion (an FcRn binding portion) of an IgG constant domain having one or more of these amino acid modifications and a non-IgG protein or non-protein molecule conjugated to such a modified IgG constant domain, wherein the presence of the modified IgG constant domain increases the in vivo half-life of the antigen binding protein.

A number of methods are known that can result in increased half-life (Kuo and Aveson, (2011)), including amino acid modifications may be generated through techniques including alanine scanning mutagenesis, random mutagenesis and screening to assess the binding to FcRn and/or the in vivo behaviour. Computational strategies followed by mutagenesis may also be used to select one of amino acid mutations to mutate.

The present invention therefore provides a variant of an antigen binding protein with optimized binding to FcRn. In a preferred embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat.

In a further aspect of the invention, the modifications are M252Y/S254T/T256E.

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

### pH Switch Technology to Increase Half-Life

Although substitutions in the constant region are able to significantly improve the functions of therapeutic IgG antibodies, substitutions in the strictly conserved constant region have the risk of immunogenicity in human and substitution in the highly diverse variable region sequence might be less immunogenic. Reports concerned with the variable region include engineering the CDR residues to improve binding affinity to the antigen and engineering, the CDR and framework residues to improve stability and decrease immunogenicity risk. As is known, improved affinity to the antigen can be achieved by affinity maturation using the phage or ribosome display of a randomized library.

Improved stability can be rationally obtained from sequence- and structure-based rational design. Decreased immunogenicity risk (deimmunization) can be accomplished by various humanization methodologies and the removal of T-cell epitopes, which can be predicted using in silico technologies or determined by in vitro assays. Additionally, variable regions have been engineered to lower pI. A longer half-life was observed for these antibodies as compared to wild type antibodies despite comparable FcRn binding. Engineering or selecting antibodies with pH dependent antigen binding to modify antibody and/or antigen half-life eg IgG2 antibody half-life can be shortened if antigen-mediated clearance mechanisms normally degrade the antibody when bound to the antigen. Similarly, the antigen:antibody complex can impact the half-life of the antigen, either extending half-life by protecting the antigen from the typical degradation processes, or shortening the half-life via antibody-mediated degradation. One embodiment relates to antibodies with higher affinity for antigen at pH 7.4 as compared to endosomal pH (i.e., pH 5.5-6.0) such that the KD ratio at pH5.5/ pH 7.4 or at pH 6.0/ pH 7.4 is 2 or more. For example to enhance the pharmacokinetic (PK) and pharmacodynamic (PD) properties of the antibody, it is possible to engineer pH-sensitive binding to the antibody by introducing histidines into CDR residues.

Additionally, methods of producing an antigen binding protein with a decreased biological half-life are also provided. A variant IgG in which His435 is mutated to alanine results in the selective loss of FcRn binding and a significantly reduced serum half-life (see for example US6,165,745 discloses a method of producing an antigen binding protein with a decreased biological half-life by introducing a mutation into the DNA segment encoding the antigen binding protein. The mutation includes an amino acid substitution at position 253, 310, 311, 433, or 434 of the Fc-hinge domain.

### Linkers

Protein scaffolds may be the same as naturally occurring sequences, such as Ig sequences, or be fragments of naturally occurring sequences, and may contain additional sequences which may be naturally occurring, from a difference source or synthetic, and which may be added at the N or C terminus of the scaffold. Such additional sequences may be considered to be linkers when they link an epitope binding domain and protein scaffold, such as those defined herein.

In another aspect the antigen binding construct consists of, or consists essentially of, an Fc region of an antibody, or a part thereof, linked at each end, directly or indirectly (for example, via a linker sequence) to an epitope binding domain. Such an antigen binding construct may comprise 2 epitope-binding domains separated by an Fc region, or part thereof. By separated is meant that the epitope-binding domains are not directly linked to one another, and in one aspect are located at opposite ends (C and N terminus) of an Fc region, or any other scaffold region.

In one aspect the antigen binding construct comprises 2 scaffold regions each bound to 2 epitope binding domains, for example at the N and C termini of each scaffold region, either directly or indirectly via a linker.

Protein scaffolds of the present invention may be linked to epitope-binding domains by the use of linkers. Examples of suitable linkers include amino acid sequences which may be from 1 amino acid to 150 amino acids in length, or from 1 amino acid to 140 amino acids, for example, from 1 amino acid to 130 amino acids, or from 1 to 120 amino acids, or from 1 to 80 amino acids, or from 1 to 50 amino acids, or from 1 to 20 amino acids, or from 1 to 10 amino acids, or from 5 to 18 amino acids. Such sequences may have their own tertiary structure, for example, a linker of the present invention may comprise a single variable domain. The size of a linker in one embodiment is equivalent to a single variable domain. Suitable linkers may be of a size from 1 to 100 angstroms, for example may be of a size from 20 to 80 angstroms or for example may be of a size from 20 to 60 angstroms or for example less than 40 angstroms, or less than 20 angstroms, or less than 5 angstroms in length.

In one embodiment, the IL1RAP binding protein is a monoclonal antibody (mAb). Suitably the IL1RAP binding protein is a humanized monoclonal antibody. In one aspect the monoclonal antibodies of the present invention can be fully human.

In another aspect, the IL1RAP binding protein is a fragment which is a Fab, Fab′, F(ab′)₂, Fv, diabody, triabody, tetrabody, miniantibody, minibody, isolated V_{H} or isolated V_{L}. In one embodiment, the IL1RAP binding protein is an antigen binding portion thereof.

Further to the invention, are pharmaceutical compositions comprising an IL1RAP binding protein or a monoclonal antibody described herein. In one aspect the pharmaceutical composition of the present invention further comprise at least one anti-neoplastic agent. In one aspect the pharmaceutical composition of the present invention further comprise at least one second immunomodulatory agent. In one aspect, the pharmaceutical composition of the present invention further comprising at least one immunostimulatory adjuvant.

An IL1RAP binding protein of the present invention can be used to treat a solid tumor. In one aspect, the tumor is selected from head and neck cancer, gastric cancer, melanoma, renal cell carcinoma (RCC), esophageal cancer, non-small cell lung carcinoma, prostate cancer, colorectal cancer, ovarian cancer, pancreatic cancer, cervical cancer, bladder cancer, breast cancer, melanoma, renal cell carcinoma (RCC), EC squamous cell cancer, and mesothelioma. In another aspect, the human has a liquid tumor such as multiple myeloma, chronic lyphomblastic leukemia (CLL), follicular lymphoma, acute myeloid leukemia, myelodysplastic syndrome and chronic myelogenous leukemia.

By the term “treating” and grammatical variations thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, (4) to slow the progression of the condition or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the terms “cancer,” “neoplasm,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom’s macroglobulinemia; lymphomas such as non-Hodgkin’s lymphoma, Hodgkin’s lymphoma; and the like.

An IL1RAP binding protein of the present invention may be used to treat a cancer in which may be an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

The IL1RAP binding proteins of the invention can be used in conjunction with other recombinant proteins and/or peptides (such as tumor antigens or cancer cells) in order to increase an immune response to these proteins (i.e., in a vaccination protocol).

For example, IL1RAP binding proteins thereof may be used to stimulate antigen-specific immune responses by coadministration of at least one IL1RAP binding protein with an antigen of interest (e.g., a vaccine). Accordingly, in another aspect the invention provides a method of enhancing an immune response to an antigen in a subject, comprising administering to the subject: (i) the antigen; and (ii) an IL1RAP binding protein of the invention, such that an immune response to the antigen in the subject is enhanced. The antigen can be, for example, a tumor antigen, a viral antigen, a bacterial antigen or an antigen from a pathogen. Non-limiting examples of such antigens include, without limitation, tumor antigens, or antigens from the viruses, bacteria or other pathogens.

As used herein “tumor antigens” are proteins that are produced by tumor cells that elicit an immune response, particularly T-cell mediated immune responses. The term “tumor antigen” as used herein includes both tumor-specific antigens and tumor-associated antigens. Tumor-specific antigens are unique to tumor cells and do not occur on other cells in the body. Tumor-associated antigens are not unique to a tumor cell and instead is also expressed on a normal cell under conditions that fail to induce a state of immunologic tolerance to the antigen. The expression of the antigen on the tumor may occur under conditions that enable the immune system to respond to the antigen. Tumor-associated antigens may be antigens that are expressed on normal cells during fetal development when the immune system is immature and unable to respond or they may be antigens that are normally present at extremely low levels on normal cells but which are expressed at much higher levels on tumor cells.

Non-limiting examples of tumor antigens include the following: differentiation antigens such as MART-1/MelanA (MART-I), gp100 (Pmel 17), tyrosinase, TRP-1, TRP-2 and tumor-specific multilineage antigens such, MAGE family antigens including but not limited to MAGE1, MAGE3, MAGE10, MAGE11, MAGE12, MAGEA2, MAGEA3, MAGEA4, MAGEA6, MAGEA8, MAGEA9, MAGEB18, MAGEB6, MABEC1, MAGED2, MAGEE1, MAGEH1, MAGEL2, BAGE, GAGE-1, GAGE-2, p15; MEL4, melanoma associated antigen 100+, melanoma gp100, NRIP3, NYS48, OCIAD1, OFA-iLRP, OIP5, ovarian carcinoma-associated antigen (OV632), PAGE4, PARP9, PATE, plastin L, PRAME, prostate-specific antigen, proteinase 3, prostein, Reg3a, RHAMM, ROPN1, SART2, SDCCAG8, SEL1L, SEPT1, SLC45A2, SPANX, SSX5, STXGALNAC1, STEAP4, survivin, TBC1D2, TEM1, TRP1, tumor antigens of epithelial origin, XAGE1, XAGE2, WT-1; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other tumor antigens include, but are not limited to, TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, TPS, glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), ELF2M, neutrophil elastase, ephrinB2, CD19, CD20, CD22, ROR1, CD33/IL3Ra, c-Met, PSMA, Glycolipid F77, EGFRvIII, GD-2, insulin growth factor (IGF)-I, IGF-II, IGF-I receptor and mesothelin.

Typically, any anti-neoplastic agent that has activity versus a susceptible tumor being treated may be co-administered in the treatment of cancer in the present invention. Examples of such agents can be found in Cancer Principles and Practice of Oncology by V.T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present IL1RAP binding protein are anti-neoplastic agents including any chemotherapeutic agents, immuno-modulatory agents or immune-modulators and immunostimulatory adjuvants.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti -cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)-N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al., J. Am. Chem, Soc., 93:2325. 1971), who characterized its structure by chemical and X-ray crystallographic methods. One mechanism for its activity relates to paclitaxel’s capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P.W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. lntem, Med., 111 :273,1989) and for the treatment of breast cancer (Holmes et al., J. Nat. Cancer Inst., 83:1797,1991). It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994, lung cancer and malaria. Treatment of patients with paclitaxel results in bone marrow suppression (multiple cell lineages, Ignoff, R.J. et. al, Cancer Chemotherapy Pocket Guide, 1998) related to the duration of dosing above a threshold concentration (50 nM) (Kearns, C.M. et. al., Seminars in Oncology, 3(6) p.16-23, 1995).

Docetaxel, (2R,3S)- N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin’s Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin’s and non-Hodgkin’s malignant lymphomas. Alopecia and neurologic effects are the most common side effect of vincristine and to a lesser extent myelosupression and gastrointestinal mucositis effects occur.

Vinorelbine, 3’,4′-didehydro -4′-deoxy-C′-norvincaleukoblastine [R-(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intraand interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin’s disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin’s disease, and non-Hodgkin’s lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin’s Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also known as Actinomycin D, is commercially available in injectable form as COSMEGEN®. Dactinomycin is indicated for the treatment of Wilm’s tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroay-1-methoay-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi’s sarcoma. Myelosuppression is the most common dose limiting side effect of daunorubicin.

Doxorubicin, (8S, 10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R )-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4- (1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2’,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′, 2′-difluorocytidine monohydrochloride (P-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin’s lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3’,4’,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I - DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroay-1H-pyrano[3’,4’,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I - DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Rituximab is a chimeric monoclonal antibody which is sold as RITUXAN® and MABTHERA®. Rituximab binds to CD20 on B cells and causes cell apoptosis. Rituximab is administered intravenously and is approved for treatment of rheumatoid arthritis and B-cell non-Hodgkin’s lymphoma.

Ofatumumab is a fully human monoclonal antibody which is sold as ARZERRA®. Ofatumumab binds to CD20 on B cells and is used to treat chronic lymphocytic leukemia CLL; a type of cancer of the white blood cells) in adults who are refractory to treatment with fludarabine (Fludara) and alemtuzumab Campath).

Trastuzumab (HEREPTIN® is a humanized monoclonal antibody that binds to the HER2 receptor. It original indication is HER2 positive breast cancer.

Cetuximab (ERBITUX® is a chimeric mouse human antibody that inhibits epidermal growth factor receptor (EGFR).

mTOR inhibitors include but are not limited to rapamycin (FK506) and rapalogs, RAD001 or everolimus (Afinitor), CCI-779 or temsirolimus, AP23573, AZD8055, WYE-354, WYE-600, WYE-687 and Pp121.

Bexarotene is sold as Targretin® and is a member of a subclass of retinoids that selectively activate retinoid X receptors (RXRs). These retinoid receptors have biologic activity distinct from that of retinoic acid receptors (RARs). The chemical name is 4-[1-(5,6,7,8-tetrahydro-3,5,5,8,8-pentamethyl-2-naphthalenyl) ethenyl] benzoic acID Bexarotene is used to treat cutaneous T-cell lymphoma CTCL, a type of skin cancer) in people whose disease could not be treated successfully with at least one other medication.

Sorafenib marketed as Nexavar® is in a class of medications called multikinase inhibitors. Its chemical name is 4-[4-[[4-chloro-3-(trifluoromethyl)phenyl]carbamoylamino] phenoxy]-N-methylpyridine-2-carboxamide. Sorafenib is used to treat advanced renal cell carcinoma (a type of cancer that begins in the kidneys). Sorafenib is also used to treat unresectable hepatocellular carcinoma (a type of liver cancer that cannot be treated with surgery).

Examples of erbB inhibitors include lapatinib, erlotinib, and gefitinib. Lapatinib, N-(3-chloro-4-{[(3-fluorophenyl)methyl]oxy}phenyl)-6-[5-({[2-(methylsulfonyl)ethyl]amino}methyl)-2-furanyl]-4-quinazolinamine (represented by formula II, as illustrated), is a potent, oral, small-molecule, dual inhibitor of erbB-1 and erbB-2 (EGFR and HER2) tyrosine kinases that is approved in combination with capecitabine for the treatment of HER2-positive metastatic breast cancer.

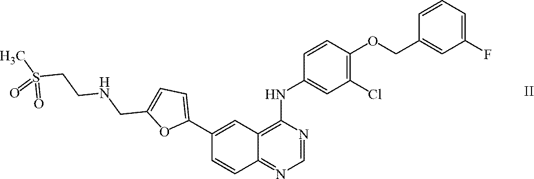 [SMILES: CS(=O)(=O)CCNCc1ccc(-c2ccc3ncnc(Nc4ccc(OCc5cccc(F)c5)c(Cl)c4)c3c2)o1]

The free base, HCl salts, and ditosylate salts of the compound of formula (II) may be prepared according to the procedures disclosed in WO 99/35146, published Jul. 15, 1999; and WO 02/02552 published Jan. 10, 2002.

Erlotinib, N-(3-ethynylphenyl)-6,7-bis{[2-(methyloxy)ethyl]oxy}-4-quinazolinamine Commercially available under the tradename Tarceva) is represented by formula III, as illustrated:

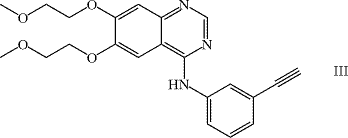 [SMILES: C#Cc1cccc(Nc2ncnc3cc(OCCOC)c(OCCOC)cc23)c1]

The free base and HCl salt of erlotinib may be prepared, for example, according to U.S. 5,747,498, Example 20.

Gefitinib, 4-quinazolinamine,N-(3-chloro-4-fluorophenyl)-7-methoxy-6-[3-4-morpholin)propoxy] is represented by formula IV, as illustrated:

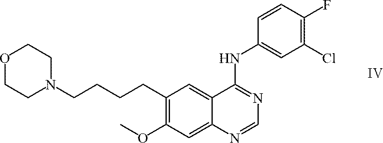 [SMILES: COc1cc2ncnc(Nc3ccc(F)c(Cl)c3)c2cc1CCCCN1CCOCC1]

Gefitinib, which is commercially available under the trade name IRESSA® (Astra-Zenenca) is an erbB-1 inhibitor that is indicated as monotherapy for the treatment of patients with locally advanced or metastatic non-small-cell lung cancer after failure of both platinum-based and docetaxel chemotherapies. The free base, HCl salts, and diHCl salts of gefitinib may be prepared according to the procedures of International Patent Application No. PCT/GB96/00961, filed Apr. 23, 1996, and published as WO 96/33980 on Oct. 31, 1996.

Also of interest, is the camptothecin derivative of formula A following, currently under development, including the racemic mixture (R,S) form as well as the R and S enantiomers:

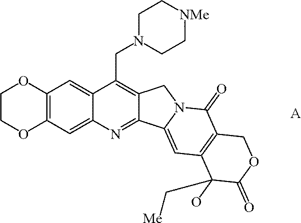 [SMILES: CCC1(O)C(=O)OCc2c1cc1n(c2=O)Cc2c-1nc1cc3c(cc1c2CN1CCN(C)CC1)OCCO3]

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. Pat. Application No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocortIL1RAPteroids such as prednisone and prednisolone which are useful in the treatment of malignant lymphoma and acute leukemia in children ; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagonists such as goserelin acetate and luprolide.

Signal transduction pathway inhibitors are those inhibitors, which block or inhibit a chemical process which evokes an intracellular change. As used herein this change is cell proliferation or differentiation. Signal transduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor -I (IGFI) receptor, macrophage colony stimulating factor Cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S.J., (1999) Journal of Hematotherapy and Stem Cell Research 8 (5): 465 - 80; and Bolen, J.B., Brugge, J.S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T.E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P.A., and Harris, A.L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R.T. (1996), Current Opinion in Immunology. 8 (3) 412-8; Canman, C.E., Lim, D.S. (1998), Oncogene 17 (25) 3301-3308; Jackson, S.P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994 New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O.G., Rozados, V.R., Gervasoni, S.I. Matar, P. (2000), Journal of Biomedical Science. 7(4 292-8; Ashby, M.N. (1998), Current Opinion in Lipidology. 9 (2) 99 - 102; and Bennett, C.F. and Cowsert, L.M. BioChim. Biophys. Acta, (1999) 1489(1):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M.C. et al, Monoclonal Antibody Therapy for Solid Tumors, Cancer Treat. Rev., (2000), 26(4):269-286); Herceptin® erbB2 antibody (see Tyrosine Kinase Signalling in Breast cancer:erbB Family Receptor Tyrosine Kniases, Breast cancer Res., 2000, 2(3):176-183); and 2CB VEGFR2 specific antibody (see Brekken, R.A. et al, Selective Inhibition of VEGFR2 Activity by a monoclonal Anti-VEGF antibody blocks tumor growth in mice, Cancer Res. (2000) 60:5117-5124.

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed erb family inhibitors. (See Bruns CJ et al (2000), Cancer Res., 60:2926-2935; Schreiber AB, Winkler ME, and Derynck R. (1986), Science, 232:1250-1253; Yen L et al. (2000), Oncogene 19:3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly RT et al. (2000), Cancer Res. 60:3569-3576; and Chen Y, Hu D, Eling DJ, Robbins J, and Kipps TJ. (1998), Cancer Res. 58:1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta’s G3139 bcl-2 antisense oligonucleotide. Such proapoptotic strategies using the antisense oligonucleotide strategy for bcl-2 are discussed in Water JS et al. (2000), J. Clin. Oncol. 18:1812-1823; and Kitada S et al. (1994, Antisense Res. Dev. 4:71-79.

Trastuzumab (HEREPTIN® is a humanized monoclonal antibody that binds to the HER2 receptor. It original indication is HER2 positive breast cancer.

Trastuzumab emtansine (trade name Kadcyla) is anantibody-drug conjugate consisting of the monoclonal antibody trastuzumab (Herceptin) linked to the cytotoxic agent mertansine (DM1). Trastuzumab alone stops growth of cancer cells by binding to the HER2/neu receptor, whereas mertansine enters cells and destroys them by binding to tubulin. Because the monoclonal antibody targets HER2, and HER2 is only over-expressed in cancer cells, the conjugate delivers the toxin specifically to tumor cells. The conjugate is abbreviated T-DM1.

Cetuximab (ERBITUX® is a chimeric mouse human antibody that inhibits epidermal growth factor receptor (EGFR).

Pertuzumab (also called 2C4, trade name Omnitarg) is a monoclonal antibody. The first of its class in a line of agents called “HER dimerization inhibitors.” By binding to HER2, it inhibits the dimerization of HER2 with other HER receptors, which is hypothesized to result in slowed tumor growth. Pertuzumab is described in WO01/00245 published Jan. 4, 2001.

Rituximab is a chimeric monoclonal antibody which is sold as RITUXAN® and MABTHERA®. Rituximab binds to CD20 on B cells and causes cell apoptosis. Rituximab is administered intravenously and is approved for treatment of rheumatoid arthritis and B-cell non-Hodgkin’s lymphoma.

Ofatumumab is a fully human monoclonal antibody which is sold as ARZERRA®. Ofatumumab binds to CD20 on B cells and is used to treat chronic lymphocytic leukemia (CLL; a type of cancer of the white blood cells) in adults who are refractory to treatment with fludarabine (Fludara) and alemtuzumab (Campath).

Cell cycle signalling inhibitors inhibit molecules involved in the control of the cell cycle. A family of protein kinases called cyclin dependent kinases CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

As used herein “immuno-modulators” refer to any substance including monoclonal antibodies that effects the immune system. The IL1RAP binding proteins of the present invention can be considered immune-modulators. Immuno-modulators can be used as anti-neoplastic agents for the treatment of cancer. For example, immune-modulators include, but are not limited to, anti-CTLA-4 antibodies such as ipilimumab (YERVOY) and anti-PD-1 antibodies (Opdivo/nivolumab and Keytruda/pembrolizumab). Other immuno-modulators include, but are not limited to, OX-40 antibodies, PD-L1 antibodies, LAG3 antibodies, TIM-3 antibodies, 41BB antibodies and GITR antibodies.

Yervoy (ipilimumab) is a fully human CTLA-4 antibody marketed by Bristol Myers Squibb. The protein structure of ipilimumab and methods are using are described in U.S. Pat. Nos. 6,984,720 and 7,605,238.

Opdivo/nivolumab is a fully human monoclonal antibody marketed by Bristol Myers Squibb directed against the negative immunoregulatory human cell surface receptor PD-1 (programmed death-1 or programmed cell death-1/PCD-1) with immunopotentiation activity. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-9365 58, MDX-1106, and ONO-4538. The amino acid sequence for nivolumab and methods of using and making are disclosed in U.S. Pat. No. US 8,008,449.

KEYTRUDA/pembrolizumab is an anti-PD-1 antibodies marketed for the treatment of lung cancer by Merck. The amino acid sequence of pembrolizumab and methods of using are disclosed in US Patent No. 8,168,757.

CD 134, also known as OX40, is a member of the TNFR-superfamily of receptors which is not constitutively expressed on resting naive T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels. OX-40 antibodies, OX-40 fusion proteins and methods of using them are disclosed in U.S. Pat. Nos: US 7,504,101; US 7,758,852; US 7,858,765; US 7,550,140; US 7,960,515; WO2012027328; WO2013028231.

Antibodies to PD-L1 (also referred to as CD274 or B7-H1) and methods for use are disclosed in U.S. Pat. No. 7,943,743; U.S. Pat. No. 8,383,796; US20130034559, WO2014055897, U.S. Pat. No. 8,168,179; and U.S. Pat.ent No. 7,595,048. PD-L1 antibodies are in development as immuno-modulatory agents for the treatment of cancer.

As used herein “immunostimulatory agent” refers to any agent that can stimulate the immune system. As used herein immunostimulatory agents include, but are not limited to, vaccine adjuvants.

Aminoalkyl glucosaminide phosphates (AGPs) are known to be useful as vaccine adjuvants and immunostimulatory agents for stimulating cytokine production, activating macrophages, promoting innate immune response, and augmenting antibody production in immunized animals. Aminoalkyl glucosaminide phosphates (AGPs) are synthetic ligands of the Toll-like Receptor 4 (TLR4). AGPs and their immunomodulating effects via TLR4 are disclosed in patent publications such as WO 2006/016997, WO 2001/090129, and/or U.S. Pat. No. 6,113,918 and have been reported in the literature. Additional AGP derivatives are disclosed in U.S. Pat. No. 7,129,219, U.S. Pat. No. 6,525,028 and U.S. Pat. No 6,911,434. Certain AGPs act as agonists of TLR4, while others are recognized as TLR4 antagonists.

Aminoalkyl glucosaminide phosphate compounds employed in the present invention have the structure set forth in Formula 1 as follows:

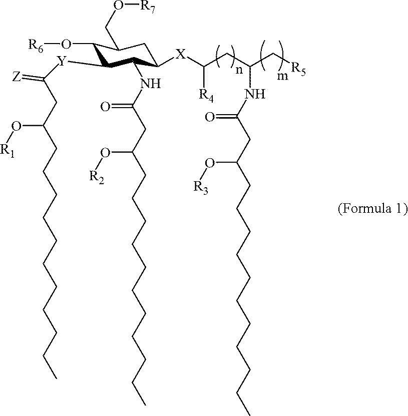

wherein
m is 0 to 6
n is 0 to 4;
X is O or S, preferably O;
Y is O or NH;
Z is O or H;
each R₁, R₂, R₃ is selected independently from the group consisting of a C₁₋₂₀ acyl and a C₁₋₂₀ alkyl;
R₄ is H or Me;
R₅ is selected independently from the group consisting of —H, —OH, -(C₁-C₄) alkoxy, -PO₃R₈R₉, -OPO₃R₈R₉, -SO₃R₈, -OSO₃R₈, -NR₈R₉, -SR₈, —CN, —NO₂, -CHO, -CO₂R₈, and -CONR₈R₉, wherein R₈ and R₉ are each independently selected from H and (C₁-C₄) alkyl; and each R₆ and R₇ is independently H or PO₃H₂.

In Formula 1 the configuration of the 3′ stereogenic centers to which the normal fatty acyl residues (that is, the secondary acyloxy or alkoxy residues, e.g., R₁O, R₂O, and R₃O) are attached is R or S, preferably R (as designated by Cahn-Ingold-Prelog priority rules). Configuration of aglycon stereogenic centers to which R₄ and R₅ are attached can be R or S. All stereoisomers, both enantiomers and diastereomers, and mixtures thereof, are considered to fall within the scope of the present invention.

The number of carbon atoms between heteroatom X and the aglycon nitrogen atom is determined by the variable “n”, which can be an integer from 0 to 4, preferably an integer from 0 to 2.

The chain length of normal fatty acids R₁, R₂, and R₃ can be from about 6 to about 16 carbons, preferably from about 9 to about 14 carbons. The chain lengths can be the same or different. Some preferred embodiments include chain lengths where R1, R2 and R3 are 6 or 10 or 12 or 14.

Formula 1 encompasses L/D-seryl, -threonyl, -cysteinyl ether and ester lipid AGPs, both agonists and antagonists and their homologs (n=1-4), as well as various carboxylic acid bioisosteres (i.e, R₅ is an acidic group capable of salt formation; the phosphate can be either on 4- or 6- position of the glucosamine unit, but preferably is in the 4-position).

In a preferred embodiment of the invention employing an AGP compound of Formula 1, n is 0, R₅ is CO₂H, R₆ is PO₃H₂, and R₇ is H. This preferred AGP compound is set forth as the structure in Formula 1a as follows:

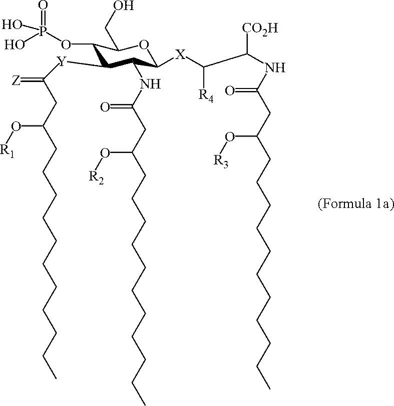

wherein X is O or S; Y is O or NH; Z is O or H; each R₁, R₂, R₃ is selected independently from the group consisting of a C₁₋₂₀ acyl and a C₁₋₂₀ alkyl; and R₄ is H or methyl.

In Formula 1a the configuration of the 3′ stereogenic centers to which the normal fatty acyl residues (that is, the secondary acyloxy or alkoxy residues, e.g., R₁O, R₂O, and R₃O) are attached as R or S, preferably R (as designated by Cahn-Ingold-Prelog priority rules). Configuration of aglycon stereogenic centers to which R₄ and CO₂H are attached can be R or S. All stereoisomers, both enantiomers and diastereomers, and mixtures thereof, are considered to fall within the scope of the present invention.

Formula 1a encompasses L/D-seryl, -threonyl, -cysteinyl ether or ester lipid AGPs, both agonists and antagonists.

In both Formula 1 and Formula 1a, Z is O attached by a double bond or two hydrogen atoms which are each attached by a single bond. That is, the compound is ester-linked when Z═Y═O; amide-linked when Z = O and Y = NH; and ether-linked when Z = H/H and Y =O.

Especially preferred compounds of Formula 1 are referred to as CRX-601 and CRX-527. Their structures are set forth as follows:

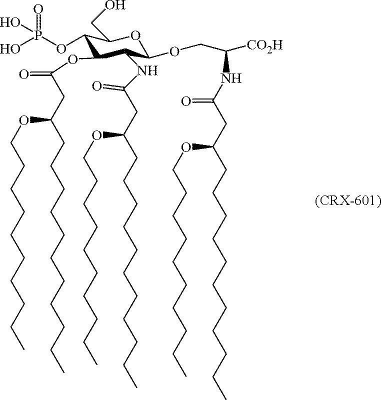

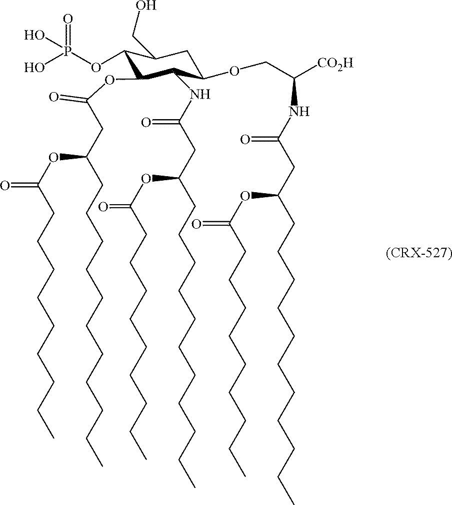

Additionally, another preferred embodiment employs CRX 547 having the structure shown. CRX 547

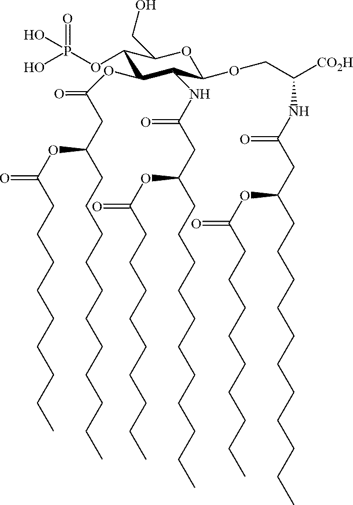

Still other embodiments include AGPs such as CRX 602 or CRX 526 providing increased stability to AGPs having shorter secondary acyl or alkyl chains.

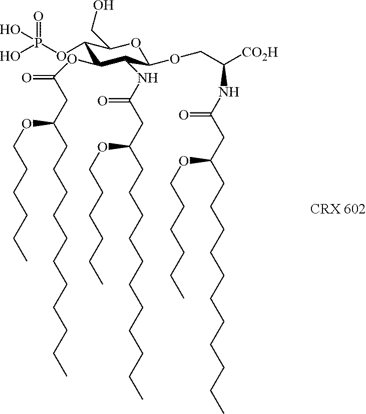

In one embodiment, methods are provided for treating cancer in a mammal in need thereof, which comprises administering to such mammal a therapeutically effective amount of:
a) an IL1RAP binding protein of the present invention, and
b) at least one anti-neoplastic agent.

In one embodiment, methods are provided for treating cancer in a mammal in need thereof, which comprises administering to such mammal a therapeutically effective amount of:
a) an IL1RAP binding protein of the present invention, and
b) at least one second immuno-modulatory agent.

In one embodiment, said second immune-modulatory agent is selected from the group of: an anti-CTLA4 antibody, an anti-PD-1 antibody, an anti-PD-L1 antibody, an anti-OX40 antibody, an anti-GITR antibody, and anti-41BB antibody, an anti-LAG3 antibody and an anti-TIM3 antibody.

In one embodiment methods are provided for treating cancer in a mammal in need thereof, which comprises: administering to such mammal a therapeutically effective amount of:
a) an IL1RAP binding protein or the present invention, and
b) at least one immuno-stimulatory agent.

### EXAMPLES

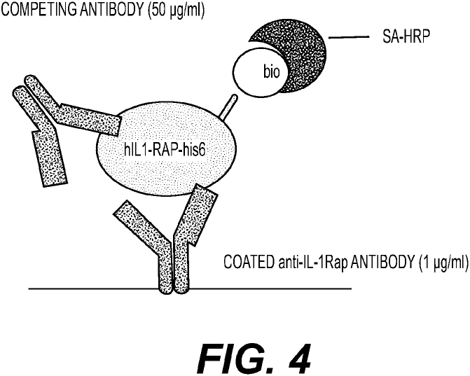

### 1. Competition Data From Binning Study (FIG. 4)

### Binning Results

Purified anti-IL1RAP mAbs were subject to competition ELISA (“binning”) experiments, in which pairs of antibodies were tested for their ability to bind human IL1RAP simultaneously. Briefly, one mAb, the capture mAb, was coated on an ELISA plate, and thereafter, a second test mAb and biotinylated IL1RAP were added. After incubation, the level of biotin-IL1RAP bound to the capture mAb was quantitated with a Streptavidin-HRP-based colorimetric assay, measured with the level of absorbance at 450 nm. If the test antibody prevents IL1RAP from binding to the capture antibody, this is reflected in a lower level of absorbance at 450 nm, and it is concluded that the two antibodies bind to the same or similar epitopes, and they are said to be in the same epitope “bin”. Based on this assay, mAb001, mAb016, mAb048, mAb063, mAb067, and mAb117 compete with each other for binding to IL1RAP and therefore thought to bind the same, or similar, epitope.

### Binning Method

Purified anti-IL1RAP antibodies were coated on 384-well ELISA plates by incubating 40 µl/well of diluted antibodies (1 µg/ml PBS) at 4° C. overnight. The plates were then washed with PBST, blocked with 40 µl blocking buffer (PBST with 2% BSA), and competing IL1RAP antibodies were added to the ELISA plate. Thereafter, a solution of biotin-labeled human IL1RAP (biotin-hIL1RAP) was added and mixed well. Competing IL1RAP antibodies were used at 10 or 50 µg/ml, and the biotin-hIL1RAP was used at a concentration identical or close to the EC50 or EC80 of the particular coated IL1RAP antibody being evaluated. Following a 1 hour incubation at 37° C., the plates were washed, and 50 µl streptavidin-peroxidase (1:5000 in wash buffer) was added to each well. The plates were incubated for 50 min at room temperature, and then washed with PBST. 50 µl TMB substrate was added to each well and incubated at room temperature for 10 min. The reaction was terminated by the addition of 50 µl 1 N HC1. Absorbance at 450 nm was measured with a spectrophotometric microplate reader. The degree to which a test antibody could compete with a capture antibody for binding to human IL1RAP was calculated by the following formula: % inhibition = (1-A450_{test mAb} /A450_{mIgG}) x 100, where mIgG is a negative control murine IgG molecule that does not bind human IL1RAP.

Competition binding table

| | | Capture mAb | | | | | |
|---|---|---|---|---|---|---|---|
| | | mAb001 | mAb016 | mAb117 | mAb048 | mAb067 | mAb063 |
| Competing mAb | mAb001 | 93% | 90% | 82% | 68% | 76% | 75% |
| | mAb016 | 95% | 94% | 89% | 78% | 76% | 80% |
| | mAb117 | 93% | 98% | 95% | 83% | 86% | 87% |
| | mAb048 | 95% | 98% | 94% | 84% | 90% | 86% |
| | mAb067 | 94% | 97% | 91% | 83% | 87% | 72% |
| | mAb063 | 94% | 97% | 92% | 82% | 87% | 96% |

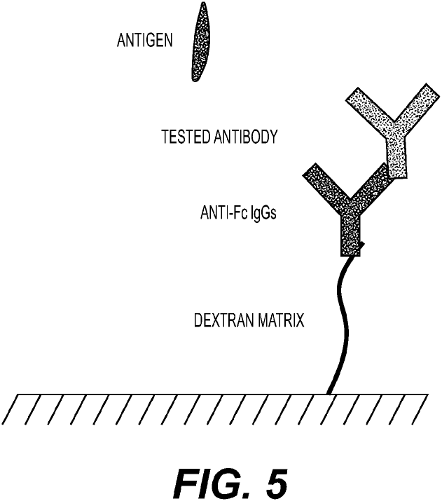

### 2. KD of All Bin 1 mAbs: mAb067, 001, 048,117, 063 (FIG. 5)

The binding kinetics and affinities of murine anti-human IL1RAP mAbs for human IL1RAP protein were determined by surface plasmon resonance technology using the Biocore T200 platform. An anti-Fc IgG antibody was immobilized on the surface of a Biacore chip and used to capture the test antibody at a flow rate of 30 µL/min. Serial dilutions of His-tagged human IL-1RAP extracellular domain protein were then flowed over the chip, with a 3-minute association phase, followed by a 20-60 minute dissociation phase. In between cycles, the Biacore chip was regenerated for 120 seconds by flowing Glycine pH 1.5 over the chip at a flow rate of 10 µL/min. Association (ka) and dissociation (kd) constants were determined, as were binding affinities (KD), with the multiple cycle kinetics algorithm.

Table of kinetic constants and binding affinities

| | | | |
|---|---|---|---|
| mAb | ka (1/Ms) | kd (1/s) | KD (M) |
| mAb001 | 5.673E+04 | 2.818E-04 | 4.968E-09 |
| mAb048 | 1.254E+05 | 4.477E-05 | 3.571E-10 |
| mAb063 | 4.263E+05 | 2.116E-05 | 4.963E-11 |
| mAb067 | 3.403E+05 | 1.021E-04 | 3.001E-10 |
| mAb117 | 2.216E+05 | 4.280E-05 | 1.932E-10 |

### 3. KD of mAb067-12 & GSK3903371A on Human IL1RAP

Surface plasmon resonance (SPR) was used to establish the binding kinetics of GSK3903371A to recombinant human and cynomolgus monkey IL1RAP. The target is observed in vivo as a membrane-bound form (mIL1RAP) and as a secreted soluble decoy receptor (sIL1RAP) formed as a result of alternatively spliced mRNA (Greenfeder 1995, Jensen 2000). A shed form of IL1RAP is also detectable from LPS-stimulated macrophages (Eichelbaum 2014). For human IL1RAP, binding kinetics were derived for recombinant purified sIL1RAP and IL1RAP extracellular domain (ECD). The first 330 amino acids of the mature cell-surface isoform (347 amino acids) and soluble IL1RAP isoform (336 amino acids) are identical. For cyno IL1RAP, binding kinetics were derived for the ECD only.

Representative kinetics for GSK3903371A binding to human and cyno IL1RAP are shown in Table 3.1 below. At 37° C. the binding affinity (KD) of GSK3903371A for human IL1RAP ECD and soluble human IL1RAP is 2.54 nM and 3.58 nM, respectively. The affinity for cyno IL1RAP ECD (geometric mean KD = 3.47 nM) meets predefined criteria for lead selection (KD for suitable non-human primate orthologue within 10-fold of KD for human IL1RAP).

### Binding Kinetics of GSK3903371A to Human and Cyno IL1RAP ECD, and to Human Soluble IL1RAP (sIL1RAP).

GSK3903371A and an irrelevant afucosylated isotype control (EPO:fIX POTELLIGENT®) were captured via Protein A on a CM5 sensor chip. Human and cyno IL1RAP ECD, and human soluble IL1RAP, were then flowed over the captured antibody to replicate non-avid target binding. Data were analyzed using a 1:1 kinetic fit model.

| | Human IL1RAP ECD | | | Soluble Human IL1RAP (sIL1RAP) | | | Cyno IL1RAP ECD | | |
|---|---|---|---|---|---|---|---|---|---|
| | Av. ka (M-1. s-1) | Av. kd (s-1) | Av. KD (nM) | Av. ka (M-1. s-1) | Av. kd (s-1) | Av. KD (nM) | Av. (M-1. s-1) | Av. kd (s-1) | Av. KD (nM) |
| 25° C. GSK3903371A | 1.70 E+05 | 2.68 E-04 | 1.61 | 1.02 E+05 | 1.99 E-04 | 2.03 | 1.49 E+05 | 2.26 E-04 | 1.53 |
| 37° C. GSK3903371A | 3.88 E+05 | 9.42 E-04 | 2.54 | 2.84 E+05 | 1.01 E-03 | 3.58 | 2.72 E+05 | 9.46 E-04 | 3.47 |

Values at 25° C. are averages of values obtained from 3 separate runs in 2 experiments
Values at 37° C. are averages of values obtained from 2 separate runs in 2 experiments

### References

Greenfeder, S.A., et al., Molecular Cloning and Characterization of a Second Subunit of the Interleukin 1 Receptor Complex. Journal of Biological Chemistry, 1995. 270(23): p. 13757-13765. Jensen, L.E., et al., IL-1 signaling cascade in liver cells and the involvement of a soluble form of the IL-1 receptor accessory protein. J Immunol, 2000. 164(10): p. 5277-86.

Eichelbaum, K. and J. Krijgsveld, Rapid Temporal Dynamics of Transcription, Protein Synthesis, and Secretion during Macrophage Activation. Molecular & Cellular Proteomics, 2014. 13(3): p. 792-810.

### KD of mAb067-12 (Humanized) on Human IL1RAP

### Determination of Binding Affinity of Humanized Antibodies by SPR (Biacore Analysis)

The binding affinity of the humanized mAb067-12 to human IL1RAP extracellular domain protein (GK002, HuIL1Racp-ECD-His6) and cyno IL1RAP (GK017, CynoIL1Racp-ECD-His6) was determined as follows. A sensor Chip CM5 (GE Healthcare, BR-1005-30) in the flow cell of a Biacore T200 (GE Healthcare) was coupled to an anti-human IgG Fc monoclonal antibody using Antibody Capture Kit (Genway, GWB-20A705). mAb067-12 was flowed over the flow cell to facilitate capture by the anti-human Fc mAb. Serial dilutions of recombinant human IL1RAP-ECD-His6 protein (GK002) or cyno IL1RAP-ECD-His6 protein (GK017) were flowed over the captured humanized mAb, and the KD was determined using Biacore T200 evaluation software v1.0.

Characterization of mAb067-12 affinity by SPR (Biacore)

| Humanized IL1RAP mAb | Affinity (Biacore) | | | | | |
|---|---|---|---|---|---|---|
| | HuIL1RAP-ECD-His6 (GK002) | | | CynoIL1RAP-ECD-His6 (GK017) | | |
| | ka (1/M·s) | kd (1/s) | K_{D} (M) | ka (1/M·s) | kd (1/s) | K_{D} (M) |
| HumAb67-12 | 1.69E+05 | 1.61E-04 | 9.53E-10 | 1.67E+05 | 1.50E-04 | 8.99E-10 |

### Detailed Methods

### Immobilization of Anti-Fc Antibodies Onto Flow Cells

Flow cells of a Series S CM5 sensor chip were activated with freshly prepared 50 mmol/L NHS and 200 mmol/L EDC buffer at a flow rate of 10 µL/min. HBS-EP+ (10 mM HEPES, 150 mM NaCl, 3 mM EDTA, 0.05% P20, pH 7.4) was used as running buffer. Anti-human Fc antibody diluted in 10 mM NaAc (pH 4.5) was injected into the activated flow cells at 10 µL/min. The remaining active coupling sites were blocked with a 420 s injection of 1 M ethanolamine.

### KD Measurement of Humanized Antibodies

Flow cell 1 was used as reference flow cell and no antibody was injected into flow cell 1. Test antibodies were captured onto one of the flow cells 2, 3 and 4 at a flow rate of 10 µL/min. Diluted GK002 or GK017 was then injected over the 4 flow cells for 180 s at 30 µL/min flow rate to collect data for association. Buffer flow was maintained for 1200 s at 30 µL/min flow rate for dissociation measurement. At the end of the dissociation measurement, the tested antibody and antigen were removed from the surface by 10 mM glycine-HCl pH 1.5 injection for 60 seconds at a flow rate of 10 µL/min. The above steps were repeated for each concentration of serially-diluted His-tagged IL1RAP ECD protein. The KD value for each antibody was evaluated using Biacore T200 evaluation software 1.0, and the data was fit with a 1:1 binding model.

### 4. mAb067-12 Was Expressed in Potelligent Cell Line to Produce GSK3903371A

GSK3903371A is a humanized monoclonal antibody directed against human interleukin-1 receptor accessory protein (IL1RAP). The functional molecule is a disulphide-linked α2β2 tetramer consisting of two light (kappa) and two heavy (IgG1) chains. The heavy chain constant region is afucosylated via expression in the POTELLIGENT™ α-1,6-fucosy1transferase (FUT8)-knockout CHO cell line (BioWa, a member of the Kyowa Hakko Kirin group). This enhances the affinity of the molecule for specific activating Fc gamma receptors (FcyRs) with a concomitant enhancement of Fc-mediated effector function (Pereira 2018). Briefly, mAb067-12 heavy and light chain expression plasmids were linearized and used to transfect CHO DG44 FUT8-/- POTELLIGENT® host cells. Drug selection was applied to enrich for growth of transfected cells, and single cell cloning was conducted to isolate a monoclonal cell line expressing GSK3903371A.

### References

Pereira, N.A., et al., The “less-is-more” in therapeutic antibodies: Afucosylated anti-cancer antibodies with enhanced antibody-dependent cellular cytotoxicity. mAbs, 2018: p. 1-44.

### 5. ADCC Data for GSK3903371A

### Example 1: Aml Cell Line, Oci-aml-1

### Description

GSK3903371A is an afucosylated Fc enhanced anti-IL1RAP antibody that is designed to potently deplete IL1RAP-expressing cells by an ADCC mechanism. GSK3903371A induced target-cell killing of OCI-AML-1 cells in three independent ADCC assays with a mean EC50 of 64.1 pM (58.5 -70.1 pM) (geometric mean/range). This EC50 was comparable with those obtained in the same assay with other batches of GSK3903371A.

### Method

Peripheral blood mononuclear cells (PBMCs) were isolated from heparinised human donor whole blood by centrifugation over Histopaque in Accuspin tubes (Sigma). PBMCs were then washed, counted, and diluted to 1xl0⁷cells/ml in target cell growth medium. The human AML cell line OCI-AML-1 was used as a target cell for ADCC assays. Europium-labeled target cells diluted to a concentration of 1×10⁵cells/ml in target cell growth medium. GSK3903371A, at a range of concentrations up to 6.7 nM, was mixed with 1 × 10⁴ target cells for 30 minutes at room temperature, prior to the addition of 5 × 10⁵ PBMC effector cells. The final ration of effector cells:target cells (E:T) was 50:1. The assay was incubated for 3 hours, and antibody-dependent cytotoxicity (ADCC) was measured by quantitating the amount of europium released from the cells.

In experiments 1 and 2, data was analysed in Excel to produce percent specific lysis values using the maximum europium release and minimum (spontaneous) europium release values from the [effector plus target only] control wells (i.e., without antibody) using the following formula: [(Experimental release - spontaneous release)/(Maximum release - spontaneous release)] × 100% In experiment 3 there was a general background increase in cell lysis with the control and test antibodies resulting in very high % cell lysis. In this instance the EC50 value was calculated using the raw fluorescence values. EC50s using the cell lysis or raw fluorescence data were comparable. EC50 values were generated using a 4-parameter logistic non-linear fit model in Grafit (Erithacus Software) using the equation:

$$\left[\text{Y=Bottom +} \left(\text{Top-Bottom}\right) / \left(1 + 10 \hat{} \left(\left(\text{LogIC50-X}\right) * \text{Hillslope}\right)\right)\right] ,$$

where X is the concentration, Y is the response and top/bottom are responses at plateaus of the curve.

Geometric Mean OCI-AML-1 target cell killing activity with GSK3903371A in a series of ADCC assays (n=3)

| Experiment | GSK3903371A EC50 (pM) | Geometric Mean (pM) |
|---|---|---|
| 1 | 63.3 | 64.1 |
| 2 | 58.9 | |
| 3 | 70.5 | |

### Example 2: Primary Aml Cells

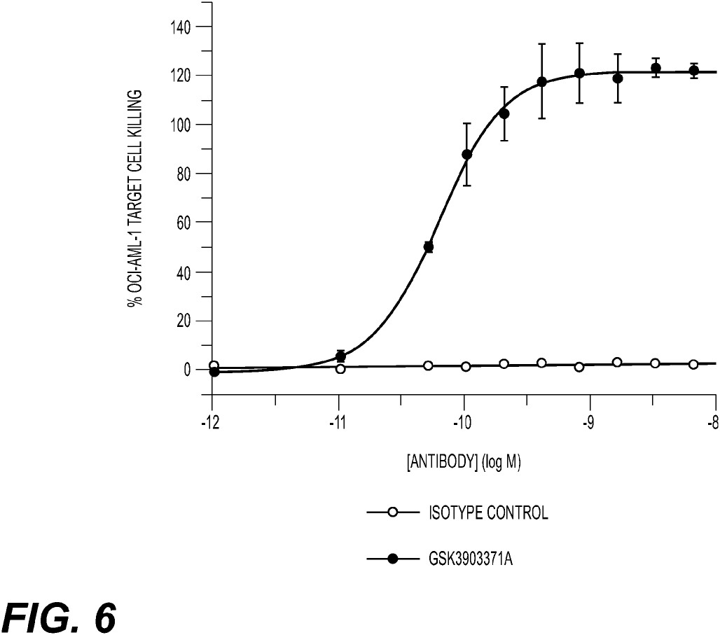

The afucosylated anti-IL1RAP mAb GSK3903371A, and the corresponding fucosylated mAb067-12, were tested in an ADCC assay against primary AML cells and human NK effector cells. GSK3903371A exhibited potent ADCC activity against primary AML cells with EC₅₀ values of 6.1, 8.8 and 10.2 pM (geometric mean 8.18 pM) in three independent experiments using NK cells from different donors (Table 1). This activity was not observed when the antibody was tested in the absence of added NK effector cells (not shown), demonstrating that the activity observed was NK mediated and not by direct lysis of the AML cells by GSK3903371A. mAb067-12 Fc WT was 10-fold less potent than GSK3903371A, with an EC₅₀ of 92 pM, as compared to GSK3903371A (EC₅₀ of 8.8 pM) (FIG. 6). mAb067-12 Fc WT exhibited a reduced maximum activity by achieving only 50% lysis of total cells defined by the control, while the maximum activity of GSK3903371A was 90% lysis of the total cells.

These data indicate that GSK3903371A has potent ADCC activity against primary AML cells and that this activity is NK-cell mediated. They also demonstrate that GSK3903371A has superior ADCC activity compared to the fucosylated version of the molecule, mAb067-12.

### Method

Serial dilutions of test antibodies were added to AML patient PBMC (target cells) at 4 × 10⁴ cells/well in RPMI1640/10% FCS, and the mixture was incubated for 30 minutes at room temperature. Healthy donor NK effector cells were added to the target cells at an effector:target (E:T) ratio of 5:1, and the cells were incubated for 18 hours at 37° C. in 5% CO2. Thereafter, cells were centrifuged at 500 g for five minutes, the supernatants were removed, and the pellets resuspended in FACS buffer. Cells were stained for the following markers in order to identify AML blasts: CD45, CD16 and/or CD337, and near IR Live/Dead™ cell stain. After staining, the cells were centrifuged, the supernatant removed, and the cell pellets were resuspended in CellFix and analysed on a flow cytometer (CytoFlex, Beckman Coulter). Standard flow cytometry analysis of forward and side scatter, as well as the Live/Dead stain, was used to identify viable single cells. A dot plot of side scatter (SSC-A) versus CD45 was utilized to identify the AML blast population. Events that were CD45 dim (considered to be the diseased blast cell population, i.e. target cell population) were gated and further plotted on a dot plot showing SSC-A versus anti-CD16 & CD337 in combination, or SSC-A versus CD16 alone, in order to exclude any NK cells. The CD45 dim, CD16 and/or CD337-negative events were gated and designated as the target cell population. The events/µL statistic for the target cell population in each test well was used to calculate percentage lysis values as detailed below.

The percentage lysis values for each test well was calculated in Excel (Microsoft) using the formula 1-(Events per µL in test well/mean of the events per µL in no antibody control wells) and presented as a percentage value. Percentage lysis values (mean of three replicates +/- SEM) were plotted in GraphPad Prism software (ver 5.0.4). EC50 values were calculated using the ‘log (agonist) vs. response - variable slope (four parameters)′ equation in GraphPad Prism.

TABLE 1

GSK3903371A ADCC EC50 against primary AML target cells (combined data for two GSK3903371A batches)

| NK cell donor | GSK3903371A batch identifier | EC₅₀ (pM) | Geometric Mean EC₅₀ (pM) |
|---|---|---|---|
| Donor A | N58072-18-11 | 6.1 | 8.18 |
| Donor B | N66353-4-4 | 10.2 | |
| Donor C | N66353-4-4 | 8.8 | |

### 6. CDC Data for GSK3903371A (and mAb067-12)

The humanized anti-IL1RAP clone mAb067-12 and its afucosylated version GSK3903371A were evaluated in complement-dependent cytotoxicity (CDC) assays, using an IL1RAP-overexpressing cell line (HEK293/IL1RAP) as target cells. Baby rabbit serum was used as a source of complement. Both HumAb067-12 and GSK3903371A exhibited potent CDC activity, with single digit nM EC50s and maximum cytotoxicity of ∼68-80%.

### Method

An HEK293 cell line clone engineered to stably-express human IL1RAP on the cell surface (HEK293/IL1RAP) was used as a target cell for assessing complement-dependent cytotoxicity (CDC) activity. HEK/IL1RAP cells were incubated with test antibody and incubated at 37° C. for 30 mins. Thereafter diluted rabbit serum was added to a concentration of 5% v/v, and the assay plates were incubated at 37° C. for 12 hrs. CellTiter-Glo Luminescent reagent was added to each well, and the plates were mixed on an orbital shaker for 2 minutes to induce cell lysis. The plate was incubated at room temperature for 10 minutes before measuring luminescence on a SpectraMax M5 microtiter plate reader. The luminescent signal is proportional to the amount of ATP released by living cells, which is used to determine the level of cytotoxicity. Data was analyzed using nonlinear regression curve fitting, evaluating the log (antibody concentration) vs. % cytotoxicity, as follows:

$$\text{\% cytotoxicity =100} \times \left(\text{E-S}\right) / \left(\text{M-S}\right)$$

where E stands for the luminescence value of the “experimental well”, S stands for the luminescence value of cells, medium, and rabbit serum (i.e., without antibody), and M stands for the luminescence value of the medium and rabbit serum (i.e., without cells and antibody).

The curve fitting model assumes that the dose-response curve has a Hill slope of 1.0, and is based on the equation

$$\text{Y=Bottom +} \left(\text{Top-Bottom}\right) / \left(1 + 10 \hat{} \left(\left(\text{LogEC50-X}\right)\right)\right)$$

Y-max, referred to as the “Max % CDC”, is the maximum lysis induced by the test antibody.

TABLE 2

CDC activity of humanized mAb067-12 and GSK3903371A

| | Experiment 1 | | Experiment 2 | |
|---|---|---|---|---|
| | Max % CDC | EC₅₀ (nM) | Max % CDC | EC₅₀ (nM) |
| mAb067-12 | 80.28 | 5.13 | 67.93 | 4.70 |
| GSK3903371A | 80.93 | 6.16 | 67.67 | 5.90 |

### 7. In Vivo Study of GSK3903371A

GSK3903371A was evaluated for the ability to inhibit the growth of a human AML PDX cell line in immunodeficient NOG mice. NOG mice were implanted with the AML PDX line LEXFAM2734 by i.v. injection. Three days later, mice were treated with 10 mg/kg GSK3903371A or isotype control by IP injection. mAbs were administered three times a week until termination of the study on day 35. GSK3903371A significantly inhibited the accumulation of human AML cells in peripheral blood after 14, 21, and 28 days. At day 35 after implantation, there was a non-significant trend for reduced AML cells in GSK3903371A-treated mice. Additionally, GSK3903371A inhibited the accumulation of AML cells in bone marrow at study termination.

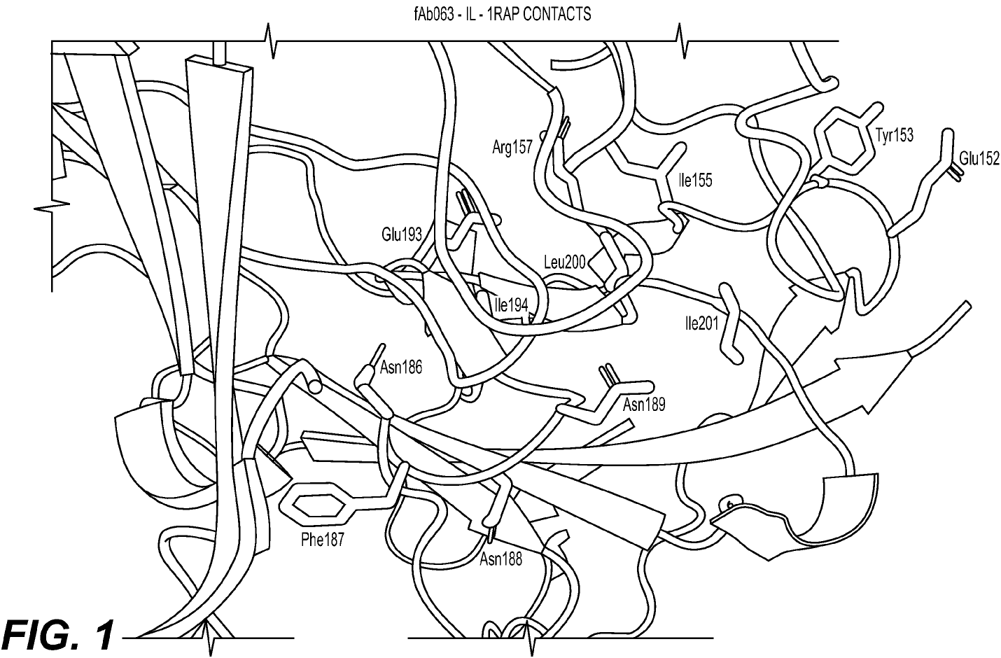

8. X-ray crystal analysis (see FIG. 1) revealed the following amino acid residues from IL1RAP protein had distances within 5 Angstrom of mAb063C-S Fab.

| | |
|---|---|
| ILE | 131 |
| GLU | 132 |
| TYR | 133 |
| GLY | 134 |
| ILE | 135 |
| ARG | 137 |
| GLN | 165 |
| ASN | 166 |
| PHE | 167 |
| ASN | 168 |
| ASN | 169 |
| VAL | 170 |
| ILE | 171 |
| PRO | 172 |
| GLU | 173 |
| SER | 178 |
| PHE | 179 |
| LEU | 180 |
| ILE | 181 |
| LEU | 183 |

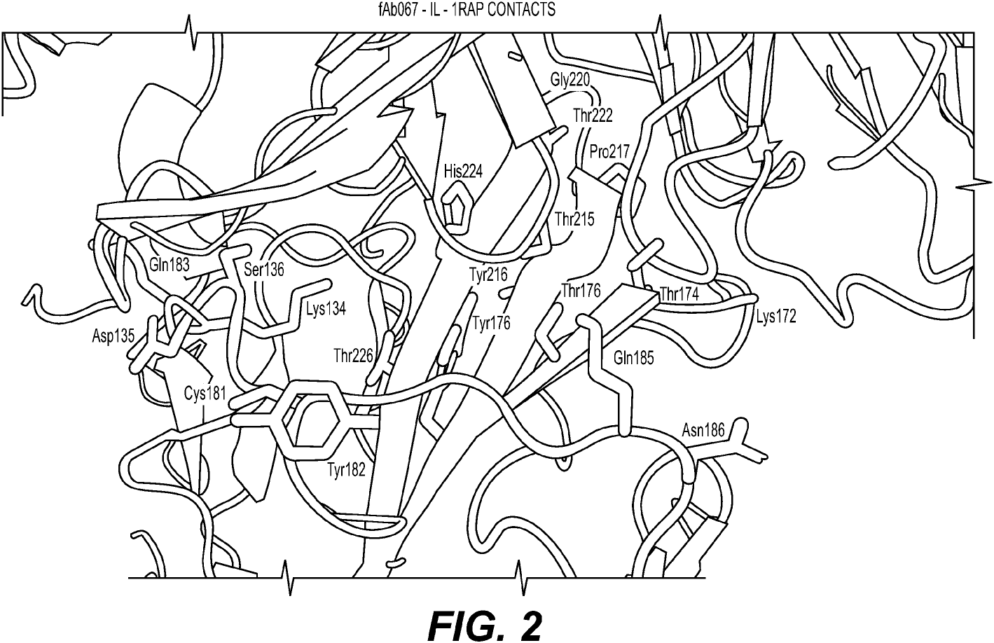

9. X-ray crystal analysis (see FIG. 2) revealed the following amino acid residues from IL1RAP protein had distances within 5 Angstrom of mAb067 Fab.

| | |
|---|---|
| GLU | 19 |
| GLU | 21 |
| GLN | 113 |
| LYS | 114 |
| ASP | 115 |
| SER | 116 |
| CYS | 117 |
| PHE | 118 |
| LYS | 152 |
| PRO | 153 |
| THR | 154 |
| ILE | 155 |
| THR | 156 |
| TYR | 158 |
| CYS | 161 |
| TYR | 162 |
| LYS | 163 |
| GLN | 165 |
| ASN | 166 |
| VAL | 193 |
| THR | 195 |
| TYR | 196 |
| PRO | 197 |
| GLY | 200 |
| ARG | 201 |
| THR | 202 |
| HIS | 204 |
| THR | 206 |

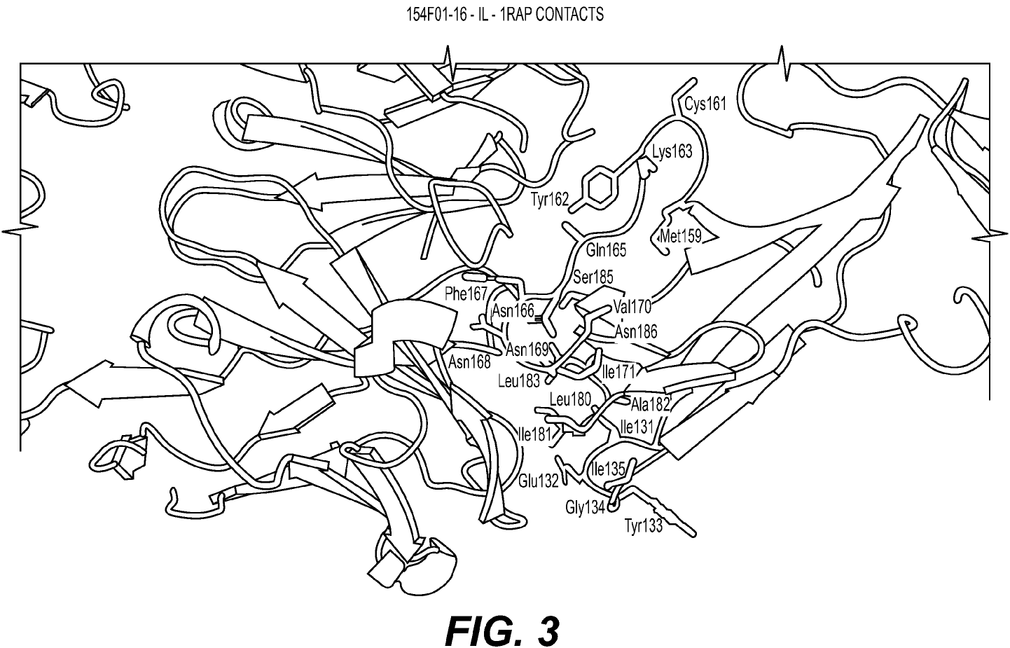

10. X-ray crystal analysis (see FIG. 3) revealed the following amino acid residues from IL1RAP protein had distances within 5 Angstrom of mAB154F01-16.

| | |
|---|---|
| ILE | 131 |
| GLU | 132 |
| TYR | 133 |
| GLY | 134 |
| ILE | 135 |
| MET | 159 |
| CYS | 161 |
| TYR | 162 |
| LYS | 163 |
| GLN | 165 |
| ASN | 166 |
| PHE | 167 |
| ASN | 168 |
| ASN | 169 |
| VAL | 170 |
| ILE | 171 |
| LEU | 180 |
| ILE | 181 |
| ALA | 182 |
| LEU | 183 |
| SER | 185 |
| ASN | 186 |

### SEQUENCE TABLE

### mAb001 (Murine)

SEQ ID NO:1 murine mAb001 heavy chain variable region cDNA sequence:

CAGGTCCAACTGCAGCAGCCTGGGGCTGAGCTGGTGAGGCCTGGGTCTTC
AGTGAAGCTGTCCTGCAAGGCTTCTGGCTACACCTTCACCAGCTACTGGA
TGGATTGGGTGAAGCAGAGGCCTGGACAAGGCCTTGAATGGATTGGTAAC
ATTTACCCTTCTGATAGTAAAACTCACTACAATCAAAAGTTCAAGGACAA
GGCCACATTGACTGTAGACAAATCCTCCAGCACAGCCTACATGCAGCTCA
GCAGCCTGACATCTGAGGACTCTGCGGTCTATTACTGTGCAAGAGATTAC
TACGGTAGCATAGGGATGGACTACTGGGGTCAAGGAACCTCAGTCACCGT
CTCCTCA

SEQ ID NO:2 Murine mAb001 heavy chain variable region protein sequence:

QVQLQQPGAELVRPGSSVKLSCKASGYTFTSYWMDWVKQRPGQGLEWIGN
IYPSDSKTHYNQKFKDKATLTVDKSSSTAYMQLSSLTSEDSAVYYCARDY
YGSIGMDYWGQGTSVTVSS

SEQ ID NO:3

SYWMD

SEQ ID NO:4

NIYPSDSKTHYNQKFKD

SEQ ID NO:5

DYYGSIGMDY

SEQ ID NO:6 Murine mAb001 light chain variable region cDNA sequence:

GACATCCAGATGACCCAGTCTCCATCCTCCTTATCTGCCTCTCTGGGAGA
AAGAGTCAGTCTCACTTGTCGGGCAAGTCAGGAAATTAGTGGTTACTTAA
GCTGGCTTCAGCAGAAACCAGATGGAACTATTAAACGCCTGATCTACGCC
GCATCCACTTTAGATTCTGGTGTCCCAAAAAGGTTCAGTGGCAGTAGGTC
TGGGTCAGATTATTCTCTCACCATTAGCAGCCTTGAGTCTGAAGATTTTG
CAGACTATTACTGTCTACAATATGCTAGTTATCCGTGGACGTTCGGTGGA
GGCACCAAGCTGGAAATCAAA

SEQ ID NO: 7 Murine mAb001 light chain variable region protein sequence:

DIQMTQSPSSLSASLGERVSLTCRASQEISGYLSWLQQKPDGTIKRLIYA
ASTLDSGVPKRFSGSRSGSDYSLTISSLESEDFADYYCLQYASYPWTFGG
GTKLEIK

SEQ ID NO:8

RASQEISGYLS

SEQ ID NO:9

AASTLDS

SEQ ID NO:10

LQYASYPWT

### Murine mAb016

SEQ ID NO: 11 Murine mAb016 heavy chain variable region cDNA sequence:

CAGGTCCAACTGCAGCAGCCTGGGGCTGAGCTGGTGAGGCCTGGGTCTTC
AGTGAAGCTGTCCTGCAAGGCTTCTGGCTACACCTTCACCAGCTACTGGA
TGGATTGGGTGAAGCAGAGGCCTGGACAAGGCCTTGAATGGATTGGTAAC
ATTTACCCTTCTGATAGTGCAACTCACTACAATCAAAAGTTCAAGGACAA
GGCCACATTGACTATAGACAAATCCTCCAGCACAGCCTACATGCAGCTCA
GCAGCCTGACATCTGAGGACTCTGCGGTCTATTACTGTGCAAGAGATTAC
TACGGTAGCATAGGGATGGACTCTTGGGGTCAAGGAACCTCTGTCACCGT
CTCCTCA

SEQ ID NO:12 Murine mAb016 heavy chain variable region protein sequence:

QVQLQQPGAELVRPGSSVKLSCKASGYTFTSYWMDWVKQRPGQGLEWIGN
IYPSDSATHYNKFKDKATLTIDKSSSTAYMQLSSLTSEDSAVYYCARDYY
GSIGMDSWGQGTSVTVSS

SEQ ID NO:13

SYWMD

SEQ ID NO:14

NIYPSDSATHYNQKFKD

SEQ ID NO:15

DYYGSIGMDS

SEQ ID NO:16 Murine mAb016 light chain variable region cDNA sequence:

GACATCCAGATGACCCAGTCTCCATCCTCCTTATCTGCCTCTCTGGGAGA
AAGAGTCAGTCTCACTTGTCGGGCAAGTCAGGAAATTAGTGATTACTTAA
GCTGGCTTCAGCAGAAACCAGATGGAACTATTAAACGCCTGATCTACGCC
GCATCCACTTTAGATTCTGGTGTCCCAAAAAGGTTCAGTGGCAGTAGGTC
TGGGTCAGATTATTCTCTCACCATCAGCAGCCTTGAGTCTGAAGATTTTG
CAGACTATTACTGTCTACAATATGCTAGTTTTCCGTGGACGTTCGGTGGA
GGCACCAAGCTGGAGATCAAA

SEQ ID NO:17 Murine mAb016 light chain variable region protein sequence:

DIQMTQSPSSLSASLGERVSLTCRASQEISDYLSWLQQKPDGTIKRLIYA
ASTLDSGVPKRFSGSRSGSDYSLTISSLESEDFADYYCLQYASFPWTFGG
GTKLEIK

SEQ ID NO:18

RASQEISDYLS

SEQ ID NO:19

AASTLDS

SEQ ID NO:20

LQYASFPWT

### Murine mAb048

SEQ ID NO:21 Murine mAb048 heavy chain variable region cDNA sequence:

CAGGTCCAACTGCAGCAGCCTGGGGCTGACCTGGTAAAGCCTGGGGCTTC
AGTGAAGTTGTCCTGCAAGGCTTATGGCTACACTTTCTTTAGTTACTGGA
TGCACTGGGTGAGGCAGAGGCCTGGACAAGGCCTTGAGTGGATTGGAATG
ATTCATCCTAATAGTGGTACCACTAACAACAATGAGAAGTTCAAGAGCAA
GGCCACACTGACTGTAGACAAATCCTTCAGTACAGCCTACATGCAACTCA
GCAGCCTGACATCTGAGGACTCTGCGGTCTATTACTGTGCGAGGGGTTAT
AGTAACTACGACTTTGACTACTGGGGCCAAGGCACCATTCTCACAGTCTC
CTCA

SEQ ID NO:22 Murine mAb048 heavy chain variable region protein sequence:

QVQLQQPGADLVKPGASVKLSCKAYGYTFFSYWMHWVRQRPGQGLEWIGM
IHPNSGTTNNNEKFKSKATLTVDKSFSTAYMOLSSLTSEDSAVYYCARGY
SNYDFDYWGOGTILTVSS

SEQ ID NO:23

SYWMH

SEQ ID NO:24

MIHPNSGTTNNNEKFKS

SEQ ID NO:25

GYSNYDFDY

SEQ ID NO:26 Murine mAb048 light chain variable region cDNA sequence:

GACATCAAGATGACCCAGTTTCCATCTTCCATGTATGCTTCTCTAGGAGA
GAGAGTCACTATCACTTGCAAGGCGAGTCAGGACATTAATACCTATTTAA
TCTGGATCCAGCAGAAACCAGGGAAATCTCCTAAGACCCTGATCTATCGT
GCAAATAGATTGGCAGATGGGGTCCCATCAAGGTTCAGTGGCAGTGGATC
TGGGCAAGATTCTTCTCTCACCATCAGCAGCCTGGAGCATGAAGATATGG
GAATTTATTATTGTCTACAGTATGATGAGTTTCCGTATACGTTCGGAGGG
GGGACCAAGCTGGAAATAAAA

SEQ ID NO:27 Murine mAb048 light chain variable region protein sequence:

DIKMTQFPSSMYASLGERVTITCKASQDINTYLIWIQQKPGKSPKTLIYR
ANRLADGVPSRFSGSGSGQDSSLTISSLEHEDMGIYYCLQYDEFPYTFGG
GTKLEIK

SEQ ID NO:28

KASQDINTYLI

SEQ ID NO:29

RANRLAD

SEQ ID NO:30

LQYDEFPYT

### Murine mAb067

SEQ ID NO:31 Murine mAb067 heavy chain variable region cDNA sequence:

CAGGTTAAACTTCACCAGTCTGGGGCTGAACTGGCAAAACCTGGGGCCTC
AGTGAAGATGTCCTGCAAGGCCTCTGGCTACACCTTTAATAGCTACTGGA
TACACTGGGTAAAACAGAGGCCTGGACAGGGTCTGGAATGGATCGGATAC
AATAATCCTACCTCTGGTTATAGTGAGTACAATCAGAAGTTCACGGACAA
GGCCACATTGAGTGCAGACAAATCTTCCAGTACAGCCTACATGCAACTGA
GCAGCCTGACATCTGAGGACTCTGCAGTCTATTACTGTGTAAGAGAGTAT
GGTGACTTATTTGCTTACTGGGGCCAAGGGACTCTGGTCACTGTCTCTGC
A

SEQ ID NO:32 Murine mAb067 heavy chain variable region protein sequence:

QVKLHQSGAELAKPGASVKMSCKASGYTFNSYWIHWVKQRPGQGLEWIGY
NNPTSGYSEYNQKFTDKATLSADKSSSTAYMQLSSLTSEDSAVYYCVREY
GDLFAYWGQGTLUTVSA

SEQ ID NO:33

SYWIH

SEQ ID NO:34

YNNPTSGYSEYNQKFTD

SEQ ID NO:35

EYGDLFAY

SEQ ID NO:36 Murine mAb067 light chain variable region cDNA sequence:

AACATTGTAATGACCCAATCTCCCAAATCCATGTCCATGTCAGTAGGAGA
GAGGGTCACCTTGAGCTGCAAGGCCAGTGAGAATGTGGGTTATTCTGTAT
CCTGGTTTCAGCAGAAACCAGATCAGTCTCCAAAACTGCTGATATACGGG
GCATCCAACCGGTACACTGGGGTCCCCGATCGCTTCACAGGCAGTGGATC
TGCAACAGATTTCACTCTGACCATCAGCACTGTGCAGGCTGAAGACCTTG
CAGATTATCACTGTGGACAGATTTACATCTATCCGTACACGTTCGGAGGG
GGGACCAAGCTGGAAATAAAA

SEQ ID NO:37 Murine mAb067 light chain variable region protein sequence:

NIVMTQSPKSMSMSVGERVTLSCKASENVGYSVSWFQQKPDQSPKLLIYG
ASNRYTGVPDRFTGSGSATDFTLTISTVQAEDLADYHCGQIYIYPYTFGG
GTKLEIK

SEQ ID NO:38

KASENVGYSVS

SEQ ID NO:39

GASNRYT

SEQ ID NO:40

GQIYIYPYT

### Murine mAb117

SEQ ID NO:41 Murine mAb117 heavy chain variable region cDNA sequence:

CAGGCTTATCTACAGCAGTCTGGGGCTGAGCTGGTGAGGCCTGGGGCCTC
AGTGAAGATGTCCTGCAAGGCTTCTGGCTACACATTTACCAGTTACAATA
TGCACTGGGTAAAGCAGTCACCTAGACAGGGCCTGGAATGGGTTGGAGTT
TTTTATTTAGGAAATGGTGATACTTCCTACAATGAGAAGTTCAAGGGCAA
GGCCACACTGACTGTGGACAAATCCTCCAGCACAGCCTACATGCAGCTCA
GCAGCCTGACATCTGAAGACTCTGCGGTCTATTTCTGTGCAAGACCCGGG
GGCTATGCCAACTGGTACTTCGATGTCTGGGGCACAGGGACCACGGTCAC
CGTCTCCTCA

SEQ ID NO:42 Murine mAb117 heavy chain variable region protein sequence:

QAYLQQSGAELVRPGASVKMSCKASGYTFTSYNMHWVKQSPRQGLEWVGV
FYLGNGDTSYNEKFKGKATLTVDKSSSTAYMOLSSLTSEDSAVYFCARPG
GYANWYFDVWGTGTTVTVSS

SEQ ID NO:43

SYNMH

SEQ ID NO:44

VFYLGNGDTSYNEKFKG

SEQ ID NO:45

PGGYANWYFDV

SEQ ID NO:46 Murine mAb117 light chain variable region cDNA sequence:

GACATTGTGATGACCCAGTCTCAAAAATTCATGTCCACATCAGTAGGAGA
CTGGGTCAGCGTCACCTGCAAGGCCAGTCAGAATGTGGGTACTGAGGTAT
CCTGGTATCAACAGAAACCAGGCCAATCTCCTAATACACTGATCTACTCG
GCATCCTATCGGCACAGTGGAGTCCCTGATCGCTTCACAGGCAGTGGATC
TGGGACAGAATTCACTCTCACCATCACCAATGTGCAGTCTGAAGACTTGG
CAGACTATTTCTGTCAGCAATATAACAGCTTTCCGCTCACGTTCGGTGCT
GGGACCAAGCTGGACCTGAAA

SEQ ID NO:47 Murine mAb117 light chain variable region protein sequence:

DIVMTQSQKFMSTSVGDWVSVTCKASQNVGTEVSWYQQKPGQSPNTLIYS
ASYRHSGVPDRFTGSGSGTEFTLTITNVQSEDLADYFCQQYNSFPLTFGA
GTKLDLK

SEQ ID NO:48

KASQNVGTEVS

SEQ ID NO:49

SASYRHS

SEQ ID NO:50

QQYNSFPLT

SEQ ID NO:51 Heavy Variable for mAb067-12 (humanized)

EvqlvqsgaevkkpgssvkvsckasgYtfNSYWIH wvrqapgqglewmgY
NNPTSGYSEYNQKFTD rvtitadkststaymelsslrsedtavyycarEY
GDLFAY WGQGTLVTVSS

SEQ ID NO:52 Light variable for mAb067-12 (humanized)

AIQMTQSPSSLSASVGDRVTITCKASENVGYSVS WFQQKPGKSPKLLIYG
ASNRYT GVPSRFSGSGSATDFTLTISSLQPEDFATYHCGQIYIYPYT FGG
GTKVEIK

SEQ ID NO:53 cDNAfor heavy variable for mAb067-12 (humanized)

GAGGTGCAGCTGGTGCAGAGCGGCGCCGAAGTGAAAAAGCCCGGCAGCAG
CGTGAAGGTCAGCTGCAAGGCCTCCGGGTACACCTTCAACAGCTACTGGA
TCCACTGGGTGAGGCAGGCCCCCGGCCAGGGCCTCGAGTGGATGGGCTAC
AACAACCCCACCAGCGGCTACAGCGAGTACAACCAGAAGTTCACCGACAG
GGTGACCATCACAGCCGACAAGAGCACCAGCACCGCCTACATGGAGCTGA
GCAGCCTGAGGAGCGAGGACACCGCCGTGTATTACTGCGCAAGGGAGTAC
GGCGACCTGTTCGCCTACTGGGGCCAGGGCACCCTGGTGACCGTGAGCTC
T

SEQ ID NO:54 cDNA for light variable for mAb067-12 (humanized)

GCCATCCAGATGACCCAGAGCCCTAGCAGCCTGAGCGCCAGCGTGGGAGA
CAGGGTGACCATCACCTGCAAGGCCAGCGAGAACGTGGGCTACAGCGTGA
GCTGGTTCCAGCAGAAGCCCGGCAAGAGCCCCAAGCTGCTGATCTACGGC
GCAAGCAACAGGTACACCGGCGTGCCCTCTAGGTTTAGCGGCAGCGGCAG
CGCCACCGACTTCACCCTGACCATCAGCAGCCTCCAGCCCGAGGACTTCG
CCACCTACCACTGCGGCCAGATCTACATCTACCCCTACACTTTCGGCGGC
GGCACCAAGGTGGAGATTAAG

Heavy full-length protein for mAb067-12 (humanized) SEQ ID NO:55

EvqlvqsgaevkkpgssvkvsckasgYtfNSYWIHwvrqapgqglewmgY
NNPTSGYSEYNQKFTDrvtitadkststaymelsslrsedtavyycarEY
GDLFAYwgqgtlvtvssastkgpsvfplapsskstsggtaalgclvkdyf
pepvtvswnsgaltsgvhtfpavlqssglyslssvvtvpssslgtqtyic
nvnhkpsntkvdkkvepkscdkthtcppcpapellggpsvflfppkpkdt
lmisrtpevtcvvdvshedpevkfnwyvdgvevhnaktkpreeqynstyr
vvsvltvlhqdwlngkeykckvsnkalpapiektiskakgqprepqvytl
ppsreemtknqvsltclvkgfypsdiavewesngqpennykttppvldsd
gsfflyskltvdksrwqqgnvfscsvmhealhnhytqkslslspgk

Light full Length protein for mAb067-12 (humanized) SEQ ID NO:56

AIQMTQSPSSLSASVGDRVTITCKASENVGYSVSWFQQKPGKSPKLLIYG
ASNRYTGVPSRFSGSGSATDFTLTISSLQPEDFATYHCGQIYIYPYTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Heavy full-length cDNA for mAb067-12 (humanized) SEQ ID NO:57

GAAGTGCAGCTGGTGCAGAGCGGAGCAGAAGTGAAGAAGCCCGGAAGCAG
CGTGAAGGTGTCTTGCAAGGCCAGCGGCTACACCTTCAACAGCTACTGGA
TCCATTGGGTGCGCCAGGCTCCAGGACAGGGACTCGAGTGGATGGGATAC
AACAACCCCACCAGCGGCTACAGCGAGTACAACCAGAAGTTCACCGACCG
CGTGACCATCACAGCCGATAAGAGCACCAGCACCGCCTACATGGAGCTGT
CTAGCCTGAGGAGCGAGGACACAGCCGTGTACTATTGCGCCCGGGAGTAC
GGAGACCTGTTCGCTTATTGGGGCCAGGGAACACTGGTGACAGTGTCCAG
CGCTTCGACCAAGGGCCCATCGGTCTTCCCCCTGGCACCCTCCTCCAAGA
GCACCTCTGGGGGCACAGCGGCCCTGGGCTGCCTGGTCAAGGACTACTTC
CCCGAACCGGTGACGGTGTCGTGGAACTCAGGCGCCCTGACCAGCGGCGT
GCACACCTTCCCGGCTGTCCTACAGTCCTCAGGACTCTACTCCCTCAGCA
GCGTGGTGACCGTGCCCTCCAGCAGCTTGGGCACCCAGACCTACATCTGC
AACGTGAATCACAAGCCCAGCAACACCAAGGTGGACAAGAAAGTTGAGCC
CAAATCTTGTGACAAAACTCACACATGCCCACCGTGCCCAGCACCTGAAC
TCCTGGGGGGACCGTCAGTCTTCCTCTTCCCCCCAAAACCCAAGGACACC
CTCATGATCTCCCGGACCCCTGAGGTCACATGCGTGGTGGTGGACGTGAG
CCACGAAGACCCTGAGGTCAAGTTCAACTGGTACGTGGACGGCGTGGAGG
TGCATAATGCCAAGACAAAGCCGCGGGAGGAGCAGTACAACAGCACGTAC
CGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGA
AAACCATCTCCAAAGCCAAAGGGCAGCCCCGAGAACCACAGGTGTACACC
CTGCCCCCATCCCGCGAGGAGATGACCAAGAACCAGGTCAGCCTGACCTG
CCTGGTCAAAGGCTTCTATCCCAGCGACATCGCCGTGGAGTGGGAGAGCA
ATGGGCAGCCGGAGAACAACTACAAGACCACGCCTCCCGTGCTGGACTCC
GACGGCTCCTTCTTCCTCTACAGCAAGCTCACCGTGGACAAGAGCAGGTG
GCAGCAGGGGAACGTCTTCTCATGCTCCGTGATGCATGAGGCTCTGCACA
ACCACTACACGCAGAAGAGCCTCTCCCTGTCTCCGGGTAAATGA

Light full-length cDNA for mAb067-12 (humanized) SEQ ID NO: 58

GCCATCCAGATGACCCAGTCTCCTAGCAGCCTGAGCGCCAGCGTGGGAGA
TAGAGTGACCATCACTTGCAAGGCCAGCGAGAACGTGGGCTACAGCGTGT
CTTGGTTCCAGCAGAAGCCCGGCAAGAGCCCTAAGCTGCTGATCTACGGC
GCCTCTAACAGATACACCGGCGTGCCTAGCAGATTCAGCGGCAGCGGAAG
CGCCACAGACTTCACCCTGACCATCAGCAGCCTGCAGCCAGAAGACTTCG
CCACCTACCATTGCGGCCAGATCTACATCTACCCCTACACCTTCGGCGGA
GGCACCAAGGTGGAGATCAAGCGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGTTGA

SEQ ID NO:59 IL1RAP complete amino acid sequence

1 mtllwcvvsl yfygilqsda sercddwgld tmrqiqvfed eparikcplf ehflkfnyst
61 ahsagltliw ywtrqdrdle epinfrlpen riskekdlw frptllndtg nytcmlrntt
121 ycskvafple vvqkdscfns pmklpvhkly ieygiqritc pnvdgyfpss vkptitwymg
181 cykiqnfnnv ipegmnlsfl ialisnngny tcvvtypeng rtfhltrtlt vkvvgspkna
241 vppvihspnd hvvyekepge ellipctvyf sflmdsrnev wwtidgkkpd ditidvtine
301 sishsrtede trtqilsikk vtsedlkrsy vcharsakge vakaakvkqk vpaprytvel
361 acgfgatvll vvilivvyhv ywlemvlfyr ahfgtdetil dgkeydiyvs yarnaeeeef
421 vlltlrgvle nefgyklcif drdslpggiv tdetlsfiqk srrllwlsp nyvlqgtqal
481 lelkaglenm asrgninvil vqykavketk vkelkraktv ltvikwkgek skypqgrfwk
541 qlqvampvkk sprrsssdeq glsysslknv

mAB154F01-16 Heavy chain variable region (human) SEQ ID NO: 60

QVQLVQSGAEVKKPGASVKVSCKASGYTFGSGGISWVRQAPGQGLEWMGW
ISDYNGQTNYAQKLQGRVTMTTDTSTSTAYMELRSLRSDDTAVYYCARVG
TDTWYAFDIWGQGTLVTVSS

mAB154F01-16 Light chain variable region (human) SEQ ID NO: 61

EIVLTQSPATLSLSPGERATLSCRASQSVSSYLAWYQQKPGQAPRLLIYD
ASNRATGIPARFSGSGSGTDFTLTISSLEPEDFAVYYCQQPDNLRTFGGG
TKVEIK

mAb063 heavy chain variable region cDNA sequence: SEQ ID NO:62

GAGGTCCAGCTGCAACAATCTGGACCTGAGCTGGTGAAGCCTGGGGCTTC
AGTGAAGATATCCTGTAAGGCTTCTGGATACACGTTCACTGACTACTACA
TGAACTGGGTGAAACAGAGCCATGGAAAGAGCCTTGAGTGGATTGGACAT
ATTAATCCTAACAGCGGTAGTACTACCTACACTGAGAAGTTCAAGGACAA
GGCCACATTGACTGTAGACAAGTCCTCCAGCACAGCCTACATGGAGCTCC
GCAGCCTGACATCTGACGACTCTGCAGTCTATTACTGTGTCAGAAGGATT
GGGAAGAACTGGCATTGCGATGTCTGGGGCACAGGGACCACGGTCACCGT
CTCCTCA

mAb063 heavy chain variable region protein sequence (used in the binning study of Example 1) SEQ ID NO:63

EVQLQQSGPELVKPGASVKISCKASGYTFTDYYMNWVKQSHGKSLEWIGH
INPNSGSTTYTEKFKDKATLTVDKSSSTAYMELRSLTSDDSAVYYCVRRI
GKNWHCDVWGTGTTVTVSS

SEQ ID NO:64

DYYMN

SEQ ID NO: 65

HINPNSGSTTYTEKFKD

SEQ ID NO:66

RIGKNWHCDV

SEQ ID NO: 67 mAb063 light chain variable region cDNA sequence

GACATTGTGCTGACCCAATCTCCAGCTTCTTTGGCTGTGTCTCTAGGGCA
GAGGGCCACCATCTCCTGCAGAGCCAGTGAAAGTGTCAACTTTCATGGTA
CTCATTTAATGCACTGGTACCAACAGAAACCAGGACAGGCACCCAAACTC
CTCATCTATGCTGCATCCAACCTAGATTCTGGAGTCCCTGCCAGGTTCAG
TGGCAGTGGGTCTGAGACAGACTTCACCCTCAACATCCATCCTGTGGAGG
AGGAGGATGCTGCGACCTATTTCTGTCAGCAAAGTATTGAGGATCCATTC
ACGTTCGGCTCGGGGACATACTTGGAAATAAAA

SEQ ID NO: 68 mAb063 light chain variable region protein sequence:

DIVLTQSPASLAVSLGQRATISCRASESVNFHGTHLMHWYQQKPGQAPKL
LIYAASNLDSGVPARFSGSGSETDFTLNIHPVEEEDAATYFCQQSIEDPF
TFGSGTYLEIK

SEQ ID NO:69

RASESVNFHGTHLMH

SEQ ID NO:70

AASNLDS

SEQ ID NO:71

QQSIEDPFT

Alternative cDNA for heavy variable for mAb 067-12 SEQ ID NO: 72

GAAGTGCAGCTGGTGCAGAGCGGAGCAGAAGTGAAGAAGCCCGGAAGCAG
CGTGAAGGTGTCTTGCAAGGCCAGCGGCTACACCTTCAACAGCTACTGGA
TCCATTGGGTGCGCCAGGCTCCAGGACAGGGACTCGAGTGGATGGGATAC
AACAACCCCACCAGCGGCTACAGCGAGTACAACCAGAAGTTCACCGACCG
CGTGACCATCACAGCCGATAAGAGCACCAGCACCGCCTACATGGAGCTGT
CTAGCCTGAGGAGCGAGGACACAGCCGTGTACTATTGCGCCCGGGAGTAC
GGAGACCTGTTCGCTTATTGGGGCCAGGGAACACTGGTGACAGTGTCCAG
C

Human mAb154D01-5 heavy chain variable region SEQ ID NO: 73

EVQLVQSGAEVKKPGESLKISCKGSGYSFNSRWIGWVRQMPGKGLEWMGI
IYPGDSDVRYSPSFQGQVTISADKSISTAYLQWSSLKASDTAVYYCARVG
GGILYMDVWGKGTTVTVSS

Human mAb154D01-5 light chain variable region: SEQ ID NO:74

EIVMTQSPATLSVSPGERATLSCRASQSVSSNLAWYQQKPGQAPRLLIYG
ASTRATGIPARFSGSGSGTEFTLTISSLQSEDFAVYYCQQSYIWPPITFG
GGTKVEIK

mAb063C-S heavy chain variable region protein sequence (used in the X-ray study of Example 8, FIG. 1) SEQ ID NO:75

EVQLQQSGPELVKPGASVKISCKASGYTFTDYYMNWVKQSHGKSLEWIGH
INPNSGSTTYTEKFKDKATLTVDKSSSTAYMELRSLTSDDSAVYYCVRRI
GKNWHSDVWGTGTTVTVSS

What is claimed is:

1. An IL1RAP binding protein which competes for binding to human IL1RAP with an anti-IL1RAP antibody having heavy and light chain variable regions comprising:
SEQ ID NOs:2 and 7;
SEQ ID NOs: 12 and 17;
SEQ ID NOs:22 and 27;
SEQ ID NOs:32 and 37;
SEQ ID NOs:42 and 47;
SEQ ID NOs:51 and 52; or
SEQ ID NOs:63 and 68.

2. The IL1RAP binding protein of claim 1 further having the ability to
directly kill IL1RAP expressing tumor cells via ADCC and/or CDC,
inhibit the growth promoting effect of IL-1 on cancer cells,
and/or promote an enhanced tumor-directed immune response by inhibiting the promoting effects of IL-1 on myeloid-derived suppressor cells (MDSCs).

3. An IL1RAP binding protein which binds to one or both of Gln185 and Asn186 of human IL1RAP (numbering according to SEQ ID NO:59) as determined by X-ray crystallography.

4. An IL1RAP binding protein, wherein the IL1RAP binding protein is a monoclonal antibody comprising:
CDRH1 as set forth in SEQ ID NO:33;
CDRH2 as set forth in SEQ ID NO:34;
CDRH3 as set forth in SEQ ID NO:35;
CDRL1 as set forth in SEQ ID NO:38;
CDRL2 as set forth in SEQ ID NO:39, and
CDRL3 as set forth in SEQ ID NO:40.

5. The IL1RAP binding protein of claim 4, wherein said monoclonal antibody is a humanized antibody that comprises
a heavy chain variable (V_{H}) domain comprising an amino acid sequence at least 85, 90, 95, 98, or 99% identical to SEQ ID NO:51 and/or
a light chain variable (V_{L}) domain comprising an amino acid sequence at least 85, 90, 95, 98, or 99% identical to SEQ ID NO:52.

6. (canceled)

7. The IL1RAP binding protein of claim 5, wherein the V_{H} domain comprises SEQ ID NO:51 and the V_{L} domain comprises SEQ ID NO:52.

8. The IL1RAP binding protein of claim 7, wherein said monoclonal antibody comprises a heavy chain amino acid sequence set forth in SEQ ID NO:55 and a length light chain amino acid sequence set forth in SEQ ID NO:56.

9. The IL1RAP binding protein of claim 4, wherein the IL1RAP binding protein is expressed from an α-1,6-fucosyltransferase (FUT8)-knockout CHO cell line.

10. A pharmaceutical composition comprising the IL1RAP binding protein of claim 4.

11. A method of treating acute myeloid leukemia (AML), chronic myeloid leukemia (CML), or myelodysplastic syndrome (MDS); or a solid tumor cancer selected from the group consisting of prostate, breast, lung, colon, melanoma, bladder, brain, cervical, esophageal, gastric, head/neck, kidney, liver, ovarian, lymphoma, pancreatic, and sarcomas in a human, comprising administering a therapeutically effective amount of the IL1RAP binding protein of claim 4.

12. One or more polynucleotides encoding the IL1RAP binding protein of claim 4.

13-16. (canceled)

17. The polynucleotide(s) of claim 12, comprising a nucleotide sequence that is at least 85, 90, 95, 98, or 99% identical to SEQ ID NO:53, 54, 57, 58 or 72.

18. (canceled)

19. One or more expression vectors containing the polynucleotide(s) of claim 12.

20. A host cell comprising the expression vector(s) of claim 19.

21. A method of producing an IL1RAP binding protein, comprising
culturing the host cell of claim 20 under conditions that allow expression of the IL1RAP binding protein, and
isolating the IL1RAP binding protein from the cell culture.

22. The IL1RAP binding protein of claim 4, wherein the IL1RAP binding protein is a monoclonal antibody of human IgGi subtype.

23. The IL1RAP binding protein of claim 7, wherein the monoclonal antibody comprises
a heavy chain encoded by a nucleotide sequence at least 85, 90, 95, 98, or 99% identical to SEQ ID NO:57, and
a light chain encoded by a nucleotide sequence at least 85, 90, 95, 98, or 99% identical to SEQ ID NO:58.

24. The IL1RAP binding protein of claim 9, wherein the FUT8-knockout CHO cell line is a POTELLIGENT® cell line.

25. The IL1RAP binding protein of claim 4, wherein the IL1RAP binding protein is afucosylated.
